(19) United States
(12) Patent Application Publication
(10) Pub. No.: US 2007/0258970 A1
(43) Pub. Date: Nov. 8, 2007

(54) Methods for Inhibiting Hiv and Other Viral Infections by Modulating Ceramide Metabolism

(76) Inventors: Robert Blumenthal, Bethesda, MD (US); Catherine M. Finnegan, Baltimore, MD (US)

Correspondence Address: EDWARDS ANGELL PALMER & DODGE LLP, P.O. BOX 55874, BOSTON, MA, 02205, US

(21) Appl. No.: 10/582,411

(22) PCT Filed: Dec. 9, 2004
(86) PCT No.: PCT/US04/41512

§ 371 Date: Mar. 12, 2007

Related U.S. Application Data
(60) Provisional application No. 60/528,411, filed on Dec. 9, 2003.

(51) Int. Cl. A61K 31/165; A61K 31/07; A61K 31/20; A61K 31/203; A61K 38/46; A61P 31/18; C12Q 1/00

(52) U.S. Cl. 424/94.6; 435/4; 514/559; 514/622; 514/725

(57) ABSTRACT

The present invention provides methods of preventing infection by retroviruses and methods of treatment of patients suffering from or susceptible to a viral infection. More particularly, the present invention provides methods of preventing viral infection and methods of treating retroviral infections which comprise the administration of at least one compound comprising N-4-(hydroxyphenyl)retinamide. Preferred methods of the invention are suitable for use in the treatment of patients who are HIV positive or are suffering from or are susceptible to AIDS.

### FIELD OF THE INVENTION

This invention provides methods of inhibiting viral infection and methods of treating patients suffering from or susceptible to viral infections. More particularly, the invention provides methods of inhibiting viral infection of cells by administering to a patient one or more compounds that activate ceramide biosynthesis. The invention further provides pharmaceutical compositions comprising at least one N-aryl retinamide compound which is capable of inhibition of viral infection, and the use of such pharmaceutical compositions for treating a variety of viral infections.

### BACKGROUND OF THE INVENTION

HIV entry is mediated by the sequential interaction of the viral envelope protein with CD4 and a chemokine receptor on the target cell. Several lines of evidence indicate that these interactions occur at specific plasma membrane domains on the target cell termed “rafts”. These ordered membrane domains are enriched in sphingolipids, cholesterol and glycolipids and phase separate from phospholipids in the membrane. Ceramide, a derivative of the lipid sphingosine, localizes predominantly to raft domains. Ceramide has a small hydroxy head group and two long saturated hydrophobic chains, which in addition to intermolecular hydrogen bonding allows ceramide to pack tightly in the bilayers and promote membrane rigidity. Since ceramide is a cone shaped lipid and relatively poorly hydrated it affects the structure and curvature of the membrane microdomains where it is located. Generation of ceramide is accomplished either by de novo synthesis at the endoplasmic reticulum or in cellular membranes upon the hydrolytic removal of the phosphocholine moiety of sphingomyelin via the action of sphingomyelinases. As membrane organization is critical for HIV infection it is important to investigate the role of ceramide in the entry of this neutral pH-fusing virus.

It would be desirable to have additional methods of inhibiting the ability of viral infection of cells, which may be used alone or in combination with other methods of inhibiting viral infection. It would be further desirable to have pharmaceutical compositions that could provide modulation of the activity of enzymes involved in sphingolipid metabolism which will result in the inhibition of viral entry into a host cell with minimal side effects or toxicity. It would be particularly desirable to provide pharmaceutical compositions and methods of treatment that inhibit viral infection of cells with little or no toxic side effects on the host organism.

### SUMMARY OF THE INVENTION

We now provide methods of inhibiting viral or retroviral infections by administration of at least one compound capable of disrupting viral entry into a host cell. The methods of the invention are suitable for blocking HIV-1 entry into or exit from host cells by modulation of the ceramide metabolism pathway. The methods of the invention have minimal adverse side effects and are suitable for use in combination with other HIV therapies.

More particularly, the methods provided by the invention comprise the administration of one or more compounds capable of modulating ceramide metabolism (i.e., increasing synthesis and/or decreasing degradation of ceramide). Although not wishing to be bound by theory, membrane organization is critical to HIV entry into and exit from target cells. Thus, the invention provides methods of blocking or inhibiting HIV-1 infection of new cells by perturbing membrane organization by inducing either the de novo biosynthesis of ceramide, or by activating enzymes (SMase) involved in the generation of ceramide at the plasma membrane, by inhibiting ceramide degradation or by direct incorporation of exogenous ceramide into target cell membranes. Modulating ceramide metabolism such that ceramide levels are increased results in enhanced endocytosis of virions. Viral endocytosis results in virus inactivation due to the low pH of this cellular compartment. Thus, modulating ceramide metabolism inhibits viral infectivity by diverting virions from productive fusion at the plasma membrane to a “dead-end” endocytic pathway.

In a preferred embodiment, the invention provides methods for administration of at least one one retinamide derivative capable of activating ceramide biosynthesis or a ceramide degradation inhibitor, which increase the concentration of ceramide in the cellular membranes. In addition, the present invention provides for the co-administration of therapeutically effective amounts of ceramide generating retinoids and ceramide degradation inhibitors. Compounds used in the methods of the present invention can include ceramide degradation inhibitors selected from the group consisting of glucosyl ceramide synthase inhibitors, sphingosine-1-phosphate synthesis inhibitors, protein kinase C inhibitors, and the pharmaceutically acceptable salts thereof.

In certain preferred methods of the invention, infection of cells by a virus is prevented by administration of one or more compounds capable of modulating ceramide metabolism. These compounds have several advantages:prior work using retinamide compounds for the treatment of a variety of cancer lines has demonstrated that retinamide compounds, particularly N-aryl retinamide compounds, possess minimal cytotoxic properties against normal cells, i.e., non-cancer cell lines. Inhibition of, for example, HIV-1 infection of cells would decrease the risk of HIV systemic infection.

The cell infected with a virus, such as HIV, may be e.g. a monocyte/macrophage. Preferably, a 4-HPR compound inhibits the viral attachment/entry phase of an RNA virus in a cell by about 100%, at least about 99.9%, 80%, 75%, 60%, 50%, or 40%.

The infecting virus may be any number of RNA or DNA viruses, such as for example Retroviridae, Cystoviridae, Bimaviridae, Reoviridae, Coronaviridae, Flaviviridae, Togaviridae, “Arterivirus”, Astroviridae, Caliciviridae, Picornaviridae, Potyviridae, Orthomyxoviridae, Filoviridae, Paramyxoviridae, Rhabdoviridae, Arenaviridae, and Bunyaviridae, Herpesviridae, Poxyiridae.

In another preferred embodiment, the ceramide generating enzymes inhibit the attachment/entry stage or exit of more than one variant of, for example HIV, and preferably the strain of HIV is a highly mutating strain as compared to different HIV strains. Other mutating viruses include the rapidly mutating coronavirus which is the etiological agent for Severe Acute Respiratory Syndrome. Other viruses include influenza such as Influenza A and B.

Methods of treating a patient, e.g., a mammalian patient, suffering from or is susceptible to a viral or retroviral infection, such as HIV or the like, are provided by the present invention. Typically a pharmaceutically effective dose of at least one retinamide derivative is administered to the patient in need to prevent or inhibit the ability of HIV or any other virus to infect new host cells or exit infected cells by modulating the biosynthesis of sphingolipids and or glycosphingolipids (GSLs). Thus preferred methods of the invention utilize compounds capable of selectively inhibiting ceramide metabolism which is essential to the production of sphingolipids and reducing glycosphingolipids by administering inhibitors of glycosphingolipid inhibitors, such as for example, 1-phenyl-2-hexadecanoylamino-3-morpholino-1-propanol (PMP). Glycosphingolipids play a role in HIV entry into a cell.

Thus the present invention provides methods for treating a mammal suffering from or susceptible to a viral infection, comprising administering to the mammal a therapeutically effective amount of a retinamide compound. Preferred retinamide compounds which are suitable for use in the methods and pharmaceutical compositions of the invention include optionally substituted N-aryl-retinamide compounds, or more preferably optionally substituted N-(4-hydroxyphenyl)retinamide compounds.

Preferred compounds, which are suitable for use in the methods of inhibiting or preventing retroviral, e.g., HIV infection, include for example, compounds of the formula I: 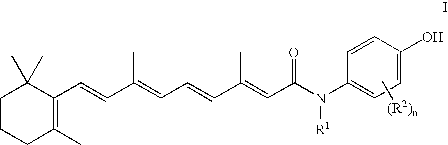
wherein:
R¹ is hydrogen, optionally substituted alkyl, optionally substituted alkenyl, optionally substituted alkynyl, or optionally substituted aralkyl;
R² is independently selected at each occurrence from the group consisting of hydrogen, halogen, hydroxy, optionally substituted alkoxy, optionally substituted alkyl, optionally substituted alkenyl, optionally substituted alkynyl, optionally substituted amino, and optionally substituted mono- and di-alkylamino; and
n is an integer of from 0 to about 4.

Preferred compounds suitable for use in the methods of the invention include those compounds of Formula I are activators of at least one enzyme essential to the ceramide metabolism and preferably are inhibitors of at least one enzyme essential to glycosylation step of ceramide metabolism. Thus the methods of the present invention prevent or inhibit attachment of a virus or HIV to the surface of a target cell or an infected cell by reducing the concentration of sphingolipids or glycosphingolipids present in the cell membrane, in addition to inhibiting membrane fusion due to the accumulation of ceramide.

The present invention further provides pharmaceutical compositions and pharmaceutical packages comprising a pharmaceutically acceptable carrier and at least one compound according to Formula I and optionally one or more additional agents suitable for the treatment or prevention of HIV or retroviral infections, including ceramide degradation inhibitors such as, glucosyl ceramide synthase inhibitors, sphingosine-1-phosphate synthesis inhibitors, protein kinase C inhibitors, and the pharmaceutically acceptable salts thereof.

Other aspects of the invention are discussed infra.

### BRIEF DESCRIPTION OF THE DRAWINGS

FIG. 1 is a schematic illustration of the ceramide metabolic pathway.

FIG. 2 is a graph showing pharmacological stimulation of ceramide biosynthesis upregulates ceramide.

FIGS. 3A and 3B are graphs showing inhibition of infection by HIV-1. FIG. 3A is a graph showing pharmacological activation of ceramide synthesis inhibits HIV-1 infection. FIG. 3B is a graph showing a 4-HPR compound inhibits infection of a broad range of HIV-1 isolates.

FIG. 4 is a graph showing sphingomyelinase activity inhibits HIV-1 infection.

FIG. 5 is a graph showing exogenous addition of ceramide inhibits HIV-1 infection.

FIG. 6A is a graph showing inhibition of HIV-1 Bal infectivity of monocyte/macrophages.

FIG. 6B is a graph showing inhibition of primary HIV isolate 92US727 infection of monocyte/macrophages.

FIG. 7 shows graphs (left panel) and FACS scans (right panel) showing a 4-HPR compound downmodulates CD4, CXCR4 and CCR5.

FIG. 8A is a graph showing that a de novo ceramide activator (a 4-HPR compound) inhibits cell-cell fusion.

FIG. 8B is a graph showing that an inhibitor of ceramide glycosylation (PPMP) in conjunction with a de novo ceramide activator (a 4-HPR compound) inhibits cell-cell fusion.

FIG. 9 is a graph showing a dose dependent inhibition of Sendai virus fusion by an activator of ceramide biosynthesis (a 4-HPR compound).

### DETAILED DESCRIPTION OF THE INVENTION

The present invention provides methods for treating a mammal suffering from or susceptible to a viral infection. In particular, methods are provided for treating and/or preventing a viral infection, such as HIV, comprising administering to the mammal a therapeutically effective amount of a N-aryl retinamide compound capable of modulating ceramide metabolism.

Prior to setting forth the invention, it may be helpful to an understanding thereof to set forth definitions of certain terms that will be used hereinafter.

“Diagnostic” means identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

As used herein, a “pharmaceutically acceptable” component is one that is suitable for use with humans and/or animals without undue adverse side effects (such as toxicity, irritation, and allergic response) commensurate with a reasonable benefit/risk ratio.

The terms “patient” or “individual” are used interchangeably herein, and is meant a mammalian subject to be treated, with human patients being preferred. In some cases, the methods of the invention find use in experimental animals, in veterinary application, and in the development of animal models for disease, including, but not limited to, rodents including mice, rats, and hamsters; and primates.

As used herein, “ameliorated” or “treatment” refers to a symptom which approaches a normalized value, e.g., is less than 50% different from a normalized value, preferably is less than about 25% different from a normalized value, more preferably, is less than 10% different from a normalized value, and still more preferably, is not significantly different from a normalized value as determined using routine statistical tests.

As used herein, “viral inhibitory activity” refers to the activity of an agent that inhibits attachment/entry, infection or any other stage of the viral life cycle as measured by a decrease in viral load or in vitro by assays that measure plaque forming units, ELISA assays and the like. The life cycle of the virus includes, attachment to a cell, penetration, uncoating of the virus particle, replication of nucleic acid sequences, production of viral capsids, encapsulation of the nucleic acid material, egress from the host cell and infection of another host cell.

As used herein, a “therapeutically effective dose” herein is meant a dose that produces the effects for which it is administered. The exact dose will depend on the purpose of the treatment, and will be ascertainable by one skilled in the art using known techniques. As is known in the art, adjustments for degradation, systemic versus localized delivery, and rate of new protease synthesis, as well as the age, body weight, general health, sex, diet, time of administration, drug interaction and the severity of the condition may be necessary, and will be ascertainable with routine experimentation by those skilled in the art.

“Cells of the immune system” or “immune cells” as used herein, is meant to include any cells of the immune system that may be assayed, including, but not limited to, B lymphocytes, also called B cells, T lymphocytes, also called T cells, natural killer (NK) cells, lymphokine-activated killer (LAK) cells, monocytes, macrophages, neutrophils, granulocytes, mast cells, platelets, Langerhans cells, stem cells, dendritic cells, peripheral blood mononuclear cells, tumor-infiltrating (TIL) cells, gene modified immune cells including hybridomas, drug modified immune cells, and derivatives, precursors or progenitors of the above cell types.

“Immune effector cells” refers to cells capable of binding an antigen and which mediate an immune response. These cells include, but are not limited to, T cells (T lymphocytes), B cells (3 lymphocytes), monocytes, macrophages, natural killer (NK) cells and cytotoxic T lymphocytes (CTLs), for example CTL lines, CTL clones, and CTLs from tumor, inflammatory, or other infiltrates.

“Immune related molecules” refers to any molecule identified in any immune cell, whether in a resting (“non-stimulated”) or activated state, and includes any receptor, ligand, cell surface molecules, nucleic acid molecules, polypeptides, variants and fragments thereof.

“T cells” or “T lymphocytes” are a subset of lymphocytes originating in the thymus and having heterodimeric receptors associated with proteins of the CD3 complex (e.g., a rearranged T cell receptor, the heterodimeric protein on the T cell surfaces responsible for antigen/MHC specificity of the cells). T cell responses may be detected by assays for their effects on other cells (e.g., target cell killing, macrophage, activation, B-cell activation) or for the cytokines they produce.

“Dendritic cells” (DC) are potent antigen-presenting cells, capable of triggering a robust adaptive immune response in vivo. It has been shown that activated, mature DC provide the signals required for T cell activation and proliferation. These signals can be categorized into two types. The first type, which gives specificity to the immune response, is mediated through interaction between the T-cell receptor/CD3 (“TCR/CD3”) complex and an antigenic peptide presented by a major histocompatibility complex (“MHC” defined above) class I or II protein on the surface of APCs. The second type of signal, called a co-stimulatory signal, is neither antigen-specific nor MHC— restricted, and can lead to a full proliferation response of T cells and induction of T cell effector functions in the presence of the first type of signals. This two-fold signaling can, therefore, result in a vigorous immune response. In most non-avian vertebrates, DC arise from bone marrow-derived precursors. Immature DC are found in the peripheral blood and cord blood and in the thymus. Additional immature populations may be present elsewhere. DC of various stages of maturity are also found in the spleen, lymph nodes, tonsils, and human intestine. Avian DC may also be found in the bursa of Fabricius, a primary immune organ unique to avians. In a preferred embodiment, the dendritic cells of the present invention are mammalian, preferably human, mouse, or rat.

“CD4” is a cell surface protein important for recognition by the T cell receptor of antigenic peptides bound to MHC class II molecules on the surface of an APC. Upon activation, naïve CD4 T cells differentiate into one of at least two cell types, Th1 cells and TH2 cells, each type being characterized by the cytokines it produces. “Th1 cells” are primarily involved in activating macrophages with respect to cellular immunity and the inflammatory response, whereas “Th2 cells” or “helper T cells” are primarily involved in stimulating B cells to produce antibodies (humoral immunity). CD4 is the receptor for the human immunodeficiency virus (HIV). Effector molecules for Th1 cells include, but are not limited to, IFN-γ, GM-CSF, TNF-α, CD40 ligand, Fas ligand, IL-3, TNF-β, and IL-2. Effector molecules for Th2 cells include, but are not limited to, IL-4, IL-5, CD40 ligand, IL-3, GS-CSF, IL-10, TGF-β, and eotaxin. Activation of the Th1 type cytokine response can suppress the Th2 type cytokine response.

As used herein, “antiviral factors” refers to any molecule produced by cells of a patient infected by a virus. For example, interferons, tumor necrosis factor, chemokines, cytokines and the like.

“CD8” is a cell surface protein important for recognition by the T cell receptor of antigenic peptides bound to MIIC class I molecules. CD8 T cells usually become “cytotoxic T cells” or “killer T cells” and activate macrophages. Effector molecules include, but are not limited to, perforin, granzymes, Fas ligand, IFN-γ, TNF-α, and TNF-β.

A “cytokine” is a protein made by a cell that affect the behavior of other cells through a “cytokine receptor” on the surface of the cells the cytokine effects. Cytokines manufactured by lymphocytes are sometimes termed “lymphokines.” Examples of cytokines include interleukins, interferons and the like.

A “chemokine” is a small cytokine involved in the migration and activation of cells, including phagocytes and lymphocytes, and plays a role in inflammatory responses. Three classes of chemokines have been defined by the arrangement of the conserved cysteine (C) residues of the mature proteins: the CXC or a chemokines that have one amino acid residue separating the first two conserved cysteine residues; the CC or β chemokines in which the first two conserved cysteine residues are adjacent; the C or γ chemokines which lack two (the first and third) of the four conserved cysteine residues. Within the CXC subfamily, the chemokines can be further divided into two groups. One group of the CXC chemokines have the characteristic three amino acid sequence ELR (glutamic acid-leucine-arginine) motif immediately preceding the first cysteine residue near the amino terminus. A second group of CXC chemokines lack such an ELR domain. The CXC chemokines with the ELR domain (including IL-8, GROα/β/γ, mouse KC, mouse MIP-2, ENA-78, GCP-2, PBP/CTAPIII/β-TG/NAP-2) act primarily on neutrophils as chemoattractants and activators, inducing neutrophil degranulation with release of myeloperoxidase and other enzymes. The CXC chemokines without the ELR domain (e.g., IP-10/mouse CRG, Mig, PBSF/SDF-1, PF4), the CC chemokines (e.g., MIP-1α, MIP-1β, RANTES, MCP-1/2/3/4/mouse JE/mouse MARC, eotaxin, I-309/TCA3, HCC-1, C10), and the C chemokines (e.g., lymphotactin), chemoattract and activate monocytes, dendritic cells, T-lymphocytes, natural killer cells, B-lymphocytes, basophils, and eosinophils.

As used herein, “chemokine receptors” refers to the cellular ligand for chemokines.

In a preferred embodiment, the de novo ceramide biosynthetic pathway is manipulated by compositions of the invention to increase plasma membrane ceramide levels, preferably by pharmacological activation of key enzymes involved in ceramide biosynthesis and decreasing the degradation of ceramide, as well as by administration of exogenous ceramide (C16 and C24) and through enzymatic cleavage of sphingomyelin at the plasma membrane. Preferably, the accumulation of ceramide in cells renders the cells resistant to viral infection, such as for example, HIV infection.

Examples of viral organisms include, but are not restricted to, those listed in table 1. For information about the viral organisms see Fields of Virology, 3. ed., vol 1 and 2, BN Fields et al. (eds.).

TABLE 1
Selected viral organisms causing human diseases.

| | |
|---|---|
| Herpesviruses | Alpha-herpesviruses: |

Herpes simplex virus 1 (HSV-1)
Herpes simplex virus 2 (HSV-2)
Varicella Zoster virus (VZV)

Beta-herpesviruses:

Cytomegalovirus (CMV)
Herpes virus 6 (HHV-6)

Gamma-herpesviruses:

Epstein-Barr virus (EBV)
Herpes virus 8 (HHV-8)

| Hepatitis viruses | Hepatitis A virus |
|---|---|
| | Hepatitis B virus |
| | Hepatitis C virus |
| | Hepatitis D virus |
| | Hepatitis E virus |
| Retroviruses | Human Immunodeficiency 1 (HIV-1)(see |
| | Example 4) |
| Orthomyxoviruses | Influenzaviruses A, B and C |
| Paramyxoviruses | Respiratory Syncytial virus (RSV) |
| | Parainfluenza viruses (PI) |
| | Mumps virus |
| | Measles virus |
| Togaviruses | Rubella virus |
| Picornaviruses | Enteroviruses |
| | Rhinoviruses |
| | Coronaviruses |
| Papovaviruses | Human papilloma viruses (HPV) |
| | Polyomaviruses (BKV and JCV) |

Gastroenteritisviruses
Filoviridae
Bunyaviridae
Rhabdoviridae
Flaviviridae

In another preferred embodiment, a composition comprising a N-4-(hydroxyphenyl) retinamide compound, such compound sometimes referred to herein as 4-BPR, inhibits viral infection of target cells. As understood, a 4-HPR compound has a 4-hydroxyphenyl substitute on retinamide groups, as such retinamide groups are identified and discussed herein. Preferably, the concentration of a 4-HPR compound inhibits infection of cells by retroviruses, such as for example HIV with a strong potency. Preferably a 4-HPR compound inhibits infection of cells by viruses with a potency (IC₅₀) up to about 1 mM, more preferably a 4-HPR compound inhibits infection of cells by viruses with a potency (IC₅₀) of about 50 μM, even more preferred a 4-HPR compound inhibits infection of cells by retroviruses with a potency (IC₅₀) of about 25 μM, 12.5 μM, 10 μM, 5 μM, 1 μM, 0.5 μM, 0.25 μM, 0.1 μM, up to about 0.001 μM.

In another preferred embodiment a pharmaceutical composition comprising N-(4-hydroxyphenyl)retinamide or a derivative thereof decreases the viral load in a subject by about 40%, at least about 50%, 60%, 75%, 80%, 99.9%, up to about 100%.

In another preferred embodiment, a 4-BPR compound or a derivative thereof, either alone or in combination with additional anti-viral agents, is administered to a patient in need thereof, to prevent or inhibit retroviral infectivity. Because of its low toxicity in non-tumor cells, a 4-HPR compound and related compounds are particularly suitable for long-term preventative or therapeutic administration to subjects suffering from, for example, HIV infection or are at risk of contracting an HIV infection.

Preferred treatment methods of the present invention comprise the administration of at least one N-aryl retinamide compound to a patient which is capable of modulating the biosynthesis of ceraminde by activating or inhibiting at least one enzyme of the ceramide metabolic pathway. Preferred N-aryl retinamide compounds include optionally substituted N-(4-hydroxyphenyl)retinamide compounds according to Formula I.

In a preferred embodiment, a single agent, for example, a 4-HPR compound, is administered to a patient in need of such therapy. Preferably, administration of a 4-HPR compound, is used to treat an individual infected with a virus, such as HIV. While administration of a single agent is preferred, a 4-HPR compound or other N-aryl retinamide compounds include optionally substituted N-(4-hydroxyphenyl)retinamide compounds according to Formula I, can be administered with one or more additional, distinct therapeutic agents, such as for example, AZT (zidovudine), ddI, ddC, d4T, 3TC, FTC, DAPD, 1592U89 or CS92; TAT antagonists such as Ro 3-3335 and Ro 24-7429; protease inhibitors such as saquinavir, ritonavir, indinavir or AG1343 (Viracept); and other agents such as 9-(2-hydroxyethoxymethyl)guanine (acyclovir), ganciclovir or penciclovir, interferon, e.g., alpha-interferon or interleukin II.

While a 4-HPR compound or other N-aryl retinamide compounds include optionally substituted N-(4-hydroxyphenyl)retinamide compounds may be administered alone, it can also be present as part of a pharmaceutical composition in mixture with conventional excipient, preferably a pharmaceutically acceptable organic or inorganic carrier substances that is generally suitable for oral or nasal delivery as mentioned previously. However, in some cases, other modes of administration may be indicated in which case the a 4-HPR compound can be combined with a vehicle suitable for parenteral, oral or other desired administration and which do not deleteriously react with the angiogenin and are not deleterious to the recipient thereof. Suitable pharmaceutically acceptable carriers include but are not limited to water, salt solutions, alcohol, vegetable oils, polyethylene glycols, gelatin, lactose, amylose, magnesium stearate, talc, silicic acid, viscous paraffin, perfume oil, fatty acid monoglycerides and diglycerides, petroethral fatty acid esters, hydroxymethyl-cellulose, polyvinylpyrrolidone, etc. The pharmaceutical preparations can be sterilized and if desired mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously react with a 4-HPR compound.

The present invention further provides a method of preventing infection of mammalian cells by a retrovirus comprising administering to the cells a therapeutically effective amount of a N-aryl-retinamide compound. Preferably the dosage of the N-aryl retinamide is sufficient to activate the biosynthesis of ceramide and thereby increase the cell membrane concentration of ceramide. Preferably, the methods prevent infection of cells by HIV. More preferably the cells are primate or human cells. In particularly preferred methods, the infection of human or primate cells by HIV is prevented or inhibited by the methods of the invention.

In another preferred embodiment, inhibition of ceramide glycosylation, for example PPMP, preferably inhibits infection by viruses such as for example, HIV, of cells such as epithelial cells and primary monocyte derived macrophages.

In another preferred embodiment, enzymatic generation of ceramide at the plasma membrane and exogenous addition of long chain ceramides preferably inhibits, for example, cellular infection by HIV infection. The results, shown in the Examples that follow, show that ceramide is an important regulator of HIV infection. Increasing ceramide levels resulted in moderate down modulation of CD4, the primary HIV-1 receptor and substantial downmodulation of the coreceptors CXCR4 and CCR5. Activation of ceramide biosynthesis alone results in significant fusion inhibition of envelope expressing cells and target cells, and combining such treatment with an inhibitor of ceramide glycosylation (PMP) further augments ceramide accumulation and fusion inhibition. Without wishing to be bound by theory, inhibition of HIV infection is due to ceramide accumulation and inhibition of viral-cell membrane fusion.

Other potential mechanisms of action of 4-HPR compounds that may promote resistance to HIV-1 infection may include:

(1) involvement of retinoic acid receptors: a 4-HPR compound has multiple distinct cellular effects. It is structurally similar to retinoids, which mediate their effects by binding to retinoic acid receptors (RAR) and retinoic X receptors (RXR). Upon ligand binding these receptors dimerize and bind to retinoid response elements on DNA leading to transcription of target genes. Retinoid induced cellular responses are extremely diverse. Retinoids may inhibit transcription of the HIV viral genome in macrophages, thus inhibiting HIV infection. There exists a possibility that the inhibition observed in macrophages following treatment with a 4-HPR compound may be due to a similar mechanism.

(2) elevation of reactive oxygen species (ROS). Treatment with a 4-BPR compound induces an elevation in ROS in a number of cell types. ROS are mediators of apoptosis, possibly by effecting changes in signaling pathways involved inactivation of transcription factors and modulation of kinases. These effects may mediate the inhibition observed in macrophages following treatment with a 4-HPR compound.

(3) treatment with a 4-BPR compound increases free radical generation in some cells types. As macrophages are highly active metabolic cells this effect may account for the inhibition observed following treatment.

(4) 4-HPR compounds activate caspase 3, a key signaling molecule in the commitment to apoptosis. This effect may contribute to the inhibition of HIV infection observed in macrophages following treatment with a 4-HPR compound.

(5) 4-HPR compounds elevate the production of transforming growth factor. This effect may contribute to the inhibition of HIV infection observed in macrophages following treatment with a 4-HPR compound.

(6) 4-HPR compounds increase Nitric Oxide synthase expression resulting in increased nitric oxide production. This effect may contribute to the inhibition of HIV infection observed in macrophages following treatment with a 4-BPR compound.

(7) 4-HPR compounds reduce telomerase activity and insulin growth factor 1 production. These effects may contribute to the inhibition of HIV infection observed in macrophages following treatment with a 4-HPR compound.

Preferably, ceramide is increased directly at 10 minutes at 37° C. with sphingomyelinase. This enzyme cleaves sphingomyelin, a phospholipid mainly located on the outer leaflet of the plasma membrane, into ceramide. Evidence indicates that sphingomyelin is localized in preformed triton insoluble microdomains termed “rafts” in the plasma membrane, which would then be the site where ceramide is generated. Particularly, large ceramide enriched rafts form and laterally segregate from sphingomyelin rich domains altering membrane organization and perturbing HIV fusion.

Preferably, pretreatment of cells susceptible to, for example, HIV, with sphingomyelinase inhibits infection of cells by at least about 40% as compared to cells not pretreated with sphingomyelinase, more preferably pretreatment of cells with sphingomyelinase inhibits infection by of cells by at least about 50% as compared to cells not pretreated with sphingomyelinase, more preferably more preferably pretreatment of cells with sphingomyelinase inhibits infection by of cells by at least about 60%, 70%, 80%, 90%, 95%, 99% or 100% as compared to cells not pretreated with sphingomyelinase.

In another preferred embodiment, pretreatment of cells susceptible to, for example, HIV, with sphingomyelinase increases ceramide expression in these cells by at feast about 10% as compared to untreated cells, more preferably, pretreatment of cells with sphingomyelinase increases ceramide expression in these cells by at least about 20% as compared to untreated cells, even more preferably, pretreatment of cells with sphingomyelinase increases ceramide expression in these cells by at least about 30%, 40%, 50%, 60%, 70%, 80%, 90%, 95%, 99%, or 100% as compared to untreated cells.

In another preferred embodiment, pretreatment of cells susceptible to, for example, HIV, with sphingomyelinase increases ceramide expression in these cells by at least about 10% as compared to untreated cells, more preferably, pretreatment of cells with sphingomyelinase increases ceramide expression in these cells by at least about 20% as compared to untreated cells, even more preferably, pretreatment of cells with sphingomyelinase increases ceramide expression in these cells by at least about 30%, 40%, 50%, 60%, 70%, 80%, 90%, 95%, 99%, or 100% as compared to untreated cells.

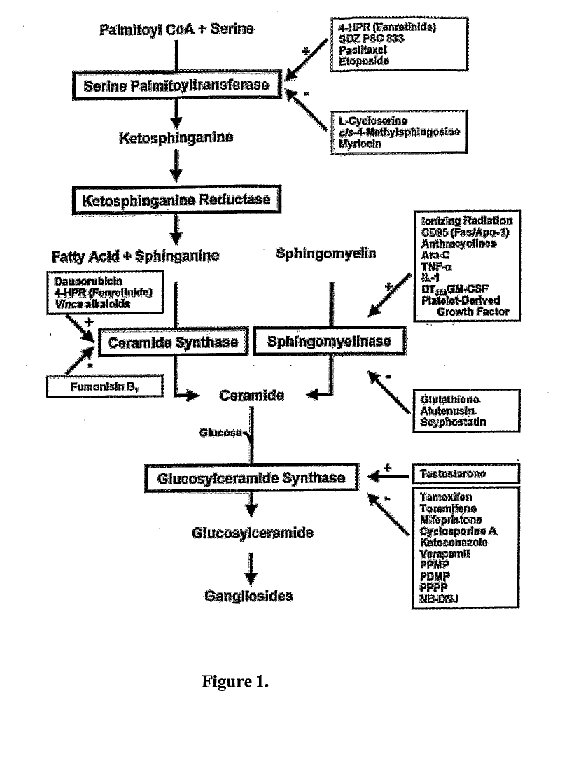

Generation of ceramide is accomplished either by de novo synthesis at the endoplasmic reticulum or in cellular membranes upon the hydrolytic removal of the phosphocholine moiety of sphingomyelin via the action of sphingomyelinases. De novo synthesis is initiated by the condensation of serine and palmitoly-CoA via serine palmitoyl-transferase as shown in FIG. 1. This reaction yields keto-sphinganine, which is reduced to form sphinganine. N-acetylation by ceramide synthase then forms dihydroceramide, which upon desaturation yields ceramide.

The invention also provides a method of reducing, preventing or delaying onset of a viral infection in a mammal comprising administering to a mammal an effective amount of a N-aryl retinamide compound or more preferably an optionally substituted N-(4-hydroxyphenyl)retinamide. More preferred methods include those wherein the retroviral infection is HIV and the mammal is HIV positive or has been exposed to HIV.

The present invention further provides a package comprising a pharmaceutical composition of a N-aryl retinamide compound compound capable of activating ceramide biosynthesis or inhibiting ceramide glycosolation and (glyco)sphingolipid formation, and further comprising indicia comprising instructions for using the composition to treat a patient suffering from or susceptible to a retroviral infection.

In a preferred embodiment, cells susceptible to infection by a virus are treated with a pharmaceutical composition of a N-aryl retinamide compound capable of activating ceramide biosynthesis or more preferably inhibiting ceramide glycosolation and (glyco)sphingolipid formation. Preferably, the pharmaceutical composition inhibits infection of a cell, by, for example, HIV. Inhibition of infection of a cell is measured by a viral-cell fusion system as described in the Examples which follow. Briefly, the cell fusion system utilizes an indicator cell line that has been engineered to express CD4 and CCR5. As CXCR4 is endogenously expressed on this cell line it is susceptible to infection by diverse HIV isolates. Following viral fusion the LTR driven reported gene products luciferase and β-galactosidase are expressed allowing for quantitative measurement of viral infectivity as soon as 16 h post infection. Such an assay system allows for the determination of viral-cell fusion inhibition as well as inhibition of early HIV lifecycle events.

Inhibition of infection of a cell can also be measured by the lack of replication of a virus, such as for example HIV. Replication of, for example, HIV can be measured using a p24 commercially available assay. For example, for each infection, a total of about 1×10⁴ cells in exponential growth phase are harvested and washed once with medium and pelleted. The cell pellet is then resuspended in about 1 ml of diluted HIV virus stock comprising about 10 TCID₅₀ units of virus. After adsorption at 37° C. for about 2 hours, about 10 ml of medium was added, and the cells were pelleted by centrifugation. They are then resuspended in about 15 ml of Iscove's and 10% FCS medium, and transferred into a 25 cm² flask. Duplicate infections per cell line were employed in each challenge assay, and the infected cultures are incubated at 37° C. Every other day beginning from day 2 post infection, about 0.5 ml of culture supernatant is removed from the flasks, and virus replication is monitored by measuring the production of p24 viral antigen in culture supernatant using an HIV-1 p24 antigen capture ELISA assay (CoulterImmunology, Hialeah, Fla.).

If desirable, a second agent can be given in conjunction with N-aryl retinamide, particularly when it is desirable to administer a lower dose of the second agent. Examples of a second agent include, but not limited to commonly used anti-retroviral drugs, such as reverse transcriptase inhibitors, protease inhibitors, and inhibitors of viral entry. Reverse transcriptase inhibitors can be nucleoside analogues, e.g., AZT (Zidovudine; Glaxo-Burroughs Wellcome Co., Research Triangle Park, NC), ddI (Didanosine; Bristol-Myers Squibb; Wallingford, Conn.), 3TC (Glaxo-Burroughs Wellcome), d4T (Stavudine; Bristol-Myers Squibb), or ddC (Zalcitabine; Hoffman-La Roche; Basel, Switzerland); or non-nucleoside drugs, e.g., Nevirapine (Viramune; Roxane Laboratories; Columbus, Ohio), Delaviridine (Rescriptor; Pharmacia & Upjohn; Kalamazoo, Mich.), Abacavir or Pyridinone (Merck, Sharp & Dohme; Rahway, N.J.). Protease inhibitors which can be used include, e.g., Indinavir (Crixivan; Merck; West Point, Pa.), Ritonavir (Novir; Abbott Laboratories; Abbott Park, Ill.), Saquinavir (Invirase; Roche; Palo Alto, Calif.), Nelfinavir (Agouron Pharmaceuticals; La Jolla, Calif.), and Amprenavir.

In another preferred embodiment, a pharmaceutical composition of a N-aryl retinamide compound capable of activating ceramide biosynthesis in addition to a compound that inhibits ceramide glycosolation and (glyco)sphingolipid formation (e.g. PPMP), inhibits infection of immune cells, especially for example CD4+ cells. Immune cells express a variety of cell surface molecules which can be detected with either monoclonal antibodies or polyclonal antisera. Immune cells that have undergone differentiation or activation can also be enumerated by staining for the presence of characteristic cell surface proteins by direct immunofluorescence in fixed smears of cultured cells. For example, monocytes, at whichever stage of maturity and cell differentiation can be identified by measuring cell phenotypes. The phenotypes of immune cells and any phenotypic changes can be evaluated by flow cytometry after immunofluorescent staining using monoclonal antibodies that will bind membrane proteins characteristic of various immune cell types.

In another preferred embodiment, patients, suffering from or susceptible to infection by, for example, HIV are treated with a pharmaceutical composition of a N-aryl retinamide compound capable of activating ceramide biosynthesis in addition to a compound that inhibits ceramide glycosolation and (glyco)sphingolipid formation (e.g PPMP). Particularly preferred embodiments include the delivery of exogenous ceramide lipids, including C16 and/or C24. The pharmaceutical composition above can be incorporated into a liposome, or other suitable carrier. The incorporation can be carried out according to well known liposome preparation procedures, such as sonication, extrusion, or microfluidization.

The liposomes can be made from any of the conventional synthetic or natural phospholipid liposome materials including phospholipids from natural sources such as egg, plant or animal sources such as phosphatidylcholine, phosphatidylethanolamine, phosphatidylglycerol, sphingomyelin, phosphatidylserine, or phosphatidylinositol. Synthetic phospholipids that may also be used, include, but are not limited to, dimyristoylphosphatidylcholine, dioleoylphosphatidylcholine, dipalmitoylphosphatidylcholine and distearoylphosphatidycholine, and the corresponding synthetic phosphatidylethanolamines and phosphatidylglycerols. Other additives such as cholesterol or other sterols, cholesterol hemisuccinate, glycolipids, cerebrosides, fatty acids, gangliosides, sphingolipids, 1,2-bis(oleoyloxy)-3-(trimethyl ammonio)propane (DOTAP), N-[1-(2,3-dioleoyl) propyl]-N,N,N-trimethylammonium (chloride) (DOTMA), D,L,-2,3-distearoyloxypropyl(dimethyl)-β-hydroxyethyl ammonium (acetate), glucopsychosine, or psychosine can also be added, as is conventionally known. The relative amounts of phospholipid and additives used in the liposomes may be varied if desired. The preferred ranges are from about 80 to 95 mole percent phospholipid and 5 to 20 mole percent psychosine or other additive. Cholesterol, cholesterol hemisuccinate, fatty acids or DOTAP may be used in amounts ranging from 0 to 50 mole percent. The amounts of antiviral nucleoside analogue incorporated into the lipid layer of liposomes can be varied with the concentration of their lipids ranging from about 0.01 to about 100 mole percent.

Preferably, the a pharmaceutical composition of a N-aryl retinamide compound capable of activating ceramide biosynthesis in addition to a compound (e.g. PPMP) that inhibits ceramide glycosolation and (glyco)sphingolipid formation, is incorporated into the lipids to achieve almost 100% of the composition being incorporated into the liposome.

The liposomes with the above formulations may be made still more specific for their intended targets with the incorporation of monoclonal antibodies or other ligands specific for a target. For example, monoclonal antibodies to the CD4 (T4) receptor may be incorporated into the liposome by linkage to phosphatidylethanolamine (PE) incorporated into the liposome. As previously described, HIV will infect those cells bearing the CD4 (T4) receptor. Use of this CD4-targeted immunoliposome will, therefore, focus antiviral compound at sites which HIV might infect. Substituting another CD4 recognition protein will accomplish the same result. On the other hand, substituting monoclonal antibody to gp120 or gp41 (HIV viral coat proteins) will focus antiviral immunoliposomes at sites of currently active HIV infection and replication. Monoclonal antibodies to other viruses, such as Herpes simplex or cytomegalovirus will focus active compound at sites of infection of these viruses.

In addition to liposomes, nanoparticles can be used as a means of delivering the compounds that are useful in the methods of the invention. Nanoparticle drug delivery, utilizing degradable and absorbable polymers, provides a more efficient solution to many drug delivery challenges. Nanoparticles are generally defined as particles between 10 nanometers (nm) and 1000 nm in size, and can be either spherical or vesicular. The advantages of using polymeric nanoparticles (PNPs) in drug delivery are many, the most important being that they generally increase the stability of any volatile pharmaceutical agents and that they are easily and cheaply fabricated in large quantities by a multitude of methods. Additionally, the use of absorbable or degradable polymers, such as polyesters, provides a high degree of biocompatibility for PNP delivery systems. Furthermore, the use of PNPs allows for design of individual delivery systems for highly specific applications. Among the adaptations that can be made are surface modifications of the polymer, use of different fabrication methods, selection of a variety of pre-existing polymers or copolymers, and formulation of novel polymeric materials. This last possibility is especially exciting, as it may be possible in the future to design specific PNP delivery systems for individuals.

In another preferred embodiment, a pharmaceutical composition kit comprising i) a pharmaceutical composition of a N-aryl retinamide compound capable of activating ceramide biosynthesis in addition to a compound that inhibits ceramide glycosolation (e.g. PPMP) and (glyco)sphingolipid formation, and ii) directions for use of the pharmaceutical composition of a N-aryl retinamide compound capable of activating ceramide biosynthesis in addition to a compound (e.g. PPMP) capable of inhibiting cerantide glycosolation and (glyco)splingolipid formation, to treat against infection by a virus.

Optionally, the kit can further comprise instructions for suitable operational parameters in the form of a label or a separate insert. For example, the kit may have standard instructions informing a consumer how to dilute a pharmaceutical composition of a N-aryl retinamide compound capable of activating ceramide biosynthesis in addition to a compound (e.g. PPMP) capable of inhibiting ceramide glycosolation and (glyco)sphingolipid formation, prior to administration, the final concentration of the diluted pharmaceutical composition of a N-aryl retinamide compound capable of activating ceramide biosynthesis in addition to a compound (e.g. PPMP) capable of inhibiting ceramide glycosolation and (glyco)sphingolipid formation, doses, the amount of time between treatments; contraindications and the like. Preferably, a pharmaceutical composition of a N-aryl retinamide compound capable of activating ceramide biosynthesis in addition to a compound (e.g. PPMP) capable of inhibiting ceramide glycosolation and (glyco)sphingolipid formation, are supplied in an effective dose and in separate ampoules to provide at least about a weeks course of treatment. The course of treatment provided in a kit preferably decreases the viral load by at least about 50%, 60%, 70%, 80%, 90% or 100%. In some embodiments, the kit may further comprise instructions for suitable operation parameters in the form of a label or a separate insert.

Chemical Description and Terminology

“Retinamide,” as used herein, is intended to include 3,7-dimethyl-9-(2,6,6-trimethyl-cyclohex-1-enyl)-nona-2,4,6,8-tetraenoic acid amide and derivatives thereof. Preferred retinamide compounds which are suitable for use in the methods of the invention include N-aryl retinamides, i.e., 3,7-dimethyl-9-(2,6,6-trimethyl-cyclohex-1-enyl)-nona-2,4,6,8-tetraenoic acid arylamides. Particularly preferred N-aryl retinamide compounds include those compounds of Formula I.

Certain compounds described herein contain one or more asymmetric elements such as stereogenic centers, stereogenic axes and the like (e.g., asymmetric carbon atoms) so that the compounds can exist in different stereoisomeric forms. These compounds can be, for example, racemates or optically active forms. For compounds with two or more asymmetric elements, these compounds can additionally be mixtures of diastereomers. Unless otherwise specified all optical isomers and mixtures thereof are encompassed for compounds having asymmetric centers. In addition, compounds with carbon-carbon double bonds may occur in Z- and E-forms, with all isomeric forms of the compounds being included in the present invention unless otherwise specified. Where a compound exists in various tautomeric forms, the invention is not limited to any one of the specific tautomers, but rather encompasses all tautomeric forms.

The present invention is intended to include all isotopes of atoms occurring in the present compounds. Isotopes include those atoms having the same atomic number but different mass numbers. By way of general example, and without limitation, isotopes of hydrogen include tritium and deuterium and isotopes of carbon include ¹¹C, ¹³C, and ¹⁴C.

Certain compounds are described herein using a general formula, such as Formula I, which includes variables, such as R₁ and R₂. Unless otherwise specified, each variable within such a formula is defined independently of other variables. Thus, for example, if a group is shown to be substituted with 0-2 R*, then said group may optionally be substituted with up to two R* groups and R* at each occurrence is selected independently from the definition of R*. Also, combinations of substituents and/or variables are permissible only if such combinations result in stable compounds.

A “derivative” as used herein, refers to those compounds identified by Formula I, comprising substitutes and optional substitutions as described below.

A “substituent,” as used herein, refers to a molecular moiety that is covalently bonded to an atom within a molecule of interest. For example, a “ring substituent” may be a moiety such as a halogen, alkyl group, haloalkyl group or other substituent discussed herein that is covalently bonded to an atom (preferably a carbon or nitrogen atom) that is a ring member. The term “substituted,” as used herein, means that any one or more hydrogens on the designated atom is replaced with a selection from the indicated substituents, provided that the designated atom's normal valence is not exceeded, and that the substitution results in a stable compound (i.e., a compound that can be isolated, characterized and tested for biological activity). When a substituent is oxo (i.e., =0), then 2 hydrogens on the atom are replaced. When aromatic moieties are substituted by an oxo group, the aromatic ring is replaced by the corresponding partially unsaturated ring. For example a pyridyl group substituted by oxo is a tetrahydropyridone.

The phrase “optionally substituted” indicates that a group may either be unsubstituted or substituted at one or more of any of the available positions, typically 1, 2, 3, 4, or 5 positions, by one or more suitable substituents such as those disclosed herein. Various groups within the compounds and formulae set forth herein are “optionally substituted” including, for example, R¹, R², and Ar¹. Optional substitution may also be indicated by the phrase “substituted with from 0 to X substituents,” in which X is the maximum number of substituents.

Suitable substituents include, for example, halogen, cyano, amino, hydroxy, nitro, azido, carboxamido, —COOH, SO₂NH₂, alkyl (e.g., C₁-C₈alkyl), alkenyl (e.g., C₂-C₈alkenyl), alkynyl(e.g., C₂-C₈alkynyl), alkoxy (e.g., C₁-C₈alkoxy), alkyl ether (e.g., C₂-C₈alkyl ether), alkylthio (e.g., C₁-C₈alkylthio), mono- or di-(C₁-C₈alkyl)amino, haloalkyl (e.g., C₁-C₆haloalkyl), hydroxyalkyl (e.g., C₁-C₆hydroxyalkyl), aminoalkyl (e.g., C₁-C₆aminoalkyl), haloalkoxy (e.g., C₁-C₆haloalkoxy), alkanoyl (e.g., C₁-C₈alkanoyl), alkanone (e.g., C₁-C₈alkanone), alkanoyloxy (e.g., C₁-C₈alkanoyloxy), alkoxycarbonyl (e.g., C₁-C₈alkoxycarbonyl), mono- and di-(C₁-C₈alkyl)amino, mono- and di-(C₁-C₈alkyl)aminoC₁-C₈alkyl, mono- and di-(C₁-C₈alkyl)carboxamido, mono- and di-(C₁-C₈alkyl)sulfonamido, alkylsulfinyl (e.g., C₁-C₈alkylsulfinyl), alkylsulfonyl (e.g., C₁-C₈alkylsulfonyl), aryl (e.g., phenyl), arylalkyl (e.g., (C₆-C₁₈aryl)C₁-C₈alkyl, such as benzyl and phenethyl), aryloxy (e.g., C₆-C₁₈aryloxy such as phenoxy), arylalkoxy (e.g., (C₆-C₁₈aryl)C₁-C₈alkoxy) and/or 3- to 8-membered heterocyclic groups. Certain groups within the formulas provided herein are optionally substituted with from 1 to 3, 1 to 4 or 1 to 5 independently selected substituents.

As used herein, “alkyl” is intended to include both branched and straight-chain saturated aliphatic hydrocarbon groups, and where specified, having the specified number of carbon atoms. Thus, the term C₁-C₆alkyl, as used herein, indicates an alkyl group having from 1 to 6 carbon atoms. “C₀-C₄alkyl” refers to a bond or a C₁-C₄alkyl group. Alkyl groups include groups having from 1 to 8 carbon atoms (C₁-C₈alkyl), from 1 to 6 carbon atoms (C₁-C₆alkyl) and from 1 to 4 carbon atoms (C₁-C₄alkyl), such as methyl, ethyl, n-propyl, isopropyl, n-butyl, sec-butyl, tert-butyl, pentyl, 2-pentyl, isopentyl, neopentyl, hexyl, 2-hexyl, 3-hexyl, and 3-methylpentyl. “Aminoalkyl” is an alkyl group as defined herein substituted with one or more —NH₂ groups. “Hydroxyalkyl” is a hydroxy group as defined herein substituted with one or more —OH groups.

“Alkenyl” refers to a straight or branched hydrocarbon chain comprising one or more unsaturated carbon-carbon bonds, such as ethenyl and propenyl. Alkenyl groups include C₂-C₈alkenyl, C₂-C₆alkenyl and C₂-C₄alkenyl groups (which have from 2 to 8, 2 to 6 or 2 to 4 carbon atoms, respectively), such as ethenyl, allyl or isopropenyl.

“Alkynyl” refers to straight or branched hydrocarbon chains comprising one or more triple carbon-carbon bonds. Alkynyl groups include C₂-C₈alkynyl, C₂-C₆alkynyl and C₂-C₄alkynyl groups, which have from 2 to 8, 2 to 6 or 2 to 4 carbon atoms, respectively. Alkynyl groups include for example groups such as ethynyl and propynyl.

“Alkoxy” represents an alkyl group as defined above with the indicated number of carbon atoms attached through an oxygen bridge. Examples of alkoxy include, but are not limited to, methoxy, ethoxy, n-propoxy, i-propoxy, n-butoxy, 2-butoxy, t-butoxy, n-pentoxy, 2-pentoxy, 3-pentoxy, isopentoxy, neopentoxy, n-hexoxy, 2-hexoxy, 3-hexoxy, and 3-methylpentoxy.

The term “alkanoyl” refers to an acyl group in a linear or branched arrangement (e.g., —(C═O)-alkyl). Alkanoyl groups include C₂-C₈alkanoyl, C₂-C₆alkanoyl and C₂-C₄alkanoyl groups, which have from 2 to 8, 2 to 6, or 2 to 4 carbon atoms, respectively. “C₁alkanoyl” refers to —(C═O)—H, which (along with C₂-C₈alkanoyl) is encompassed by the term “C₁-C₈alkanoyl.”

The term, “alkyl ether” refers to a linear or branched ether substituent linked via a carbon-carbon bond. Alkyl ether groups include C₂-C₈alkyl ether, C₂-C₆alkyl ether and C₂-C₆alkyl ether groups, which have 2 to 8, 2 to 6, or 2 to 4 carbon atoms, respectively. By way of example, a C₂alkyl ether group has the structure —CH₂—O—CH₃.

The term “alkoxycarbonyl” refers to an alkoxy group linked via a carbonyl (i.e., a group having the general structure —C(═O—O-alkyl). Alkoxycarbonyl groups include C₂-C₈, C₂-C₆, and C₂-C₄alkoxycarbonyl groups, which have from 2 to 8, 2 to 6, or 2 to 4 carbon atoms, respectively. “Calkoxycarbonyl” refers to —C(═O)OH, and is encompassed by “C₁-C₈alkoxycarbonyl.”

“Alkanoyloxy,” as used herein, refers to an alkanoyl group linked via an oxygen bridge (i.e., a group having the general structure —O—C(═O)-alkyl). Alkanoyloxy groups include C₂-C₈, C₂-C₆, and C₂-C₄alkanoyloxy groups, which have from 2 to 8, 2 to 6, or 2 to 4 carbon atoms, respectively.

As used herein, the term “alkylthio” refers to an alkyl group attached via a thioether linkage. Alkylthio groups include C₁-C₈alkylthio, C₁-C₆alkylthio and C₁-C₄alkylthio, which have from 1 to 8, 1 to 6 or 1 to 4 carbon atoms, respectively.

“Alkylsulfinyl,” as used herein, refers to an alkyl group attached via a sulfinyl linkage. Alkylsulfinyl groups include C₁-C₈alkylsulfinyl, C₁-C₆alkylsulfinyl, and C₁-C₄alkylsulfinyl, which have from 1 to 8, 1 to 6, and 1 to 4 carbon atoms, respectively.

By “alkylsulfonyl,” as used herein, is meant an alkyl group attached via a sulfonyl linkage. Alkylsulfonyl groups include C₁-C₈alkylsulfonyl, C₁-C₆alkylsulfonyl, and C₁-C₄alkylsulfonyl, which have from 1 to 8, 1 to 6, and 1 to 4 carbon atoms, respectively.

“Alkylamino” refers to a secondary or tertiary amine having the general structure —NH-alkyl or —N(alkyl)(alkyl), wherein each alkyl may be the same or different. Such groups include, for example, mono- and di-(C₁-C₈alkyl)amino groups, in which each alkyl may be the same or different and may contain from 1 to 8 carbon atoms, as well as mono- and di-(C₁-C₆alkyl)amino groups and mono- and di-(C₁-C₄alkyl)amino groups. Alkylaminoalkyl refers to an alkylamino group linked via an alkyl group (i.e., a group having the general structure -alkyl-NH-alkyl or -alkyl-N(alkyl)(alkyl)). Such groups include, for example, mono- and di-(C₁-C₈alkyl)aminoC₁-C₈alkyl, mono- and di-(C₁-C₆alkyl)aminoC-C₆alkyl, and mono- and di-(C₁-C₄alkyl)aminoC₁-C₄alkyl, in which each alkyl may be the same or different.

The term “carboxamido” or “amido” refers to an amide group (i.e., —(C═O)NH₂). “Alkylcarboxamido” refers to —NHC(═O)alkyl, preferably —NHC(═O)C₁-C₂alkyl.

The term “cycloalkyl” refers to hydrocarbon ring groups, having the specified number of carbon atoms, usually from 3 to about 8 ring carbon atoms, or from. Cycloalkyl groups include C₃-C₈, and C₃-C₇ cycloalkyl groups, which have from 3 to 8 and 3 to 7 carbon atoms, respectively. Examples of cycloalkyl groups include cyclopropyl, cyclobut-yl, cyclopentyl, and cyclohexyl groups, as well as bridged and caged saturated ring groups such as norbornane or adamantane and the like.

In the term “(cycloalkyl)alkyl,” “cycloalkyl” and “alkyl” are as defined above, and the point of attachment is on the alkyl group. This term encompasses, but is not limited to, cyclopropylmethyl, cyclohexylmethyl, and cyclohexylethyl.

The term “halogen” indicates fluorine, chlorine, bromine, or iodine.

“Haloalkyl” refers to both branched and straight-chain saturated aliphatic hydrocarbon groups having the specified number of carbon atoms, substituted with 1 or more halogen atoms. Examples of haloalkyl include, but are not limited to, trifluoromethyl, difluoromethyl, fluoromethyl, 2-fluoroethyl, and penta-fluoroethyl.

“Haloalkoxy” indicates a haloalkyl group as defined above attached through an oxygen bridge.

As used herein, the term “aryl” indicates aromatic groups containing only carbon in the aromatic ring(s). Such aromatic groups may be further substituted with carbon or non-carbon atoms or groups. Typical aryl groups contain 1 to 3 separate or fused rings, at least one of which is aromatic, and from 6 to about 18 ring atoms, without heteroatoms as ring members. Specifically preferred carbocyclic aryl groups include phenyl and naphthyl, including 1-naphthyl and 2-naphthyl. When indicated, carbon atoms present within a carbocyclic ring may be optionally substituted with any of variety of ring substituents, as described above, or with specifically listed substituents.

The term “arylalkyl” or “aralkyl” refers to an aryl group is linked via an alkyl group. Certain arylalkyl groups are (C₆-C₁₈aryl)C₁-C₈alkyl groups (i.e., groups in which a 6- to 18-membered aryl group is linked via a C₁-C₈alkyl group). Such groups include, for example, groups in which phenyl or naphthyl is linked via a bond or C₁-C₈alkyl, preferably via C₁-C₄alkyl, such as benzyl, 1-phenyl-ethyl, 1-phenyl-propyl and 2-phenyl-ethyl.

The term “aryloxy” refers to an aryl group linked via a carbonyl (i.e., a group having the general structure —C(═O)—O-aryl). Phenoxy is a representative aryloxy group.

As used herein, the term “heteroaryl” is intended to indicate a stable 5- to 7-membered monocyclic or bicyclic or 7- to 10-membered bicyclic heterocyclic ring which contains at least 1 aromatic ring that contains from 1 to 4 heteroatoms selected from N, O, and S, with remaining ring atoms being carbon. When the total number of S and O atoms in the heteroaryl group exceeds 1, then these heteroatoms are not adjacent to one another. It is preferred that the total number of S and O atoms in the heterocycle is not more than 1, 2, or 3, more typically 1 or 2. It is particularly preferred that the total number of S and O atoms in the aromatic heterocycle is not more than 1. Examples of heteroaryl groups include pyridyl, furanyl, indolyl, pyrimidinyl, pyridizinyl, pyrazinyl, imidazolyl, oxazolyl, thienyl, thiazolyl, triazolyl isoxazolyl, quinolinyl, pyrrolyl, pyrazolyl, and 5,6,7,8-tetrahydroisoquinoline.

The term “heterocyclic group” or “heterocycle” is used to indicate saturated, partially unsaturated, or aromatic groups having 1 or 2 rings, 3 to 8 atoms in each ring and in at least one ring between 1 and 3 heteroatoms selected from N, O, and S. Any nitrogen or sulfur heteroatoms may optionally be oxidized. The heterocyclic group may be attached to its pendant group at any heteroatom or carbon atom that results in a stable structure. The heterocyclic groups described herein may be substituted on a carbon or nitrogen atom if the resulting compound is stable. A nitrogen atom in the heterocycle may optionally be quaternized.

A “therapeutically effective amount” of a compound is an amount that is sufficient to result in a discernible patient benefit. For example, a therapeutically effective amount may reduce symptom severity or frequency. Alternatively, or in addition, a therapeutically effective amount may improve patient outcome and/or prevent or delay disease or symptom onset. In certain methods of the invention, a therapeutically effective amount may be capable of reducing or arresting the rate of (retro)viral replication in a mammal or a mammalian cell.

As used herein, a “pharmaceutically acceptable salt” is an acid or base salt that is generally considered in the art to be suitable for use in contact with the tissues of human beings or animals without excessive toxicity, irritation, allergic response, or other problem or complication. Such salts include mineral and organic acid salts of basic residues such as amines, as well as alkali or organic salts of acidic residues such as carboxylic acids. Specific pharmaceutical salts include, but are not limited to, salts of acids such as hydrochloric, phosphoric, hydrobromic, malic, glycolic, fumaric, sulfuric, sulfamic, sulfanilic, formic, toluenesulfonic, methanesulfonic, benzene sulfonic, ethane disulfonic, 2-hydroxyethylsulfonic, nitric, benzoic, 2-acetoxybenzoic, citric, tartaric, lactic, stearic, salicylic, glutamic, ascorbic, pamoic, succinic, fumaric, maleic, propionic, hydroxymaleic, hydroiodic, phenylacetic, alkanoic such as acetic, HOOC—(CH₂)ₙ—COOH where n is 0-4 and the like. Similarly, pharmaceutically acceptable cations include, but are not limited to sodium, potassium, calcium, aluminum, lithium and ammonium. Those of ordinary skill in the art will recognize further pharmaceutically acceptable salts for the compounds provided herein, including those listed by Remington's Pharmaceutical Sciences, 17th ed., Mack Publishing Company, Easton, Pa., p. 1418 (1985). Accordingly, the present disclosure should not be construed to include all pharmaceutically acceptable salts of the compounds specifically recited. A wide variety of synthetic procedures is available for the preparation of pharmaceutically acceptable salts. In general, a pharmaceutically acceptable salt can be synthesized from a parent compound that contains a basic or acidic moiety by any conventional chemical method. Briefly, such salts can be prepared by reacting the free acid or base forms of these compounds with a stoichiometric amount of the appropriate base or acid in water, an organic solvent, or a mixture of the two; generally, nonaqueous media like ether, ethyl acetate, ethanol, isopropanol or acetonitrile are preferred.

It will be apparent that the specific compounds recited herein are representative only, and are not intended to limit the scope of the present invention. Further, as noted above, all compounds of the present invention may be present as a lactone, a ring-opened hydrolyzed lactone or a combination thereof, or may be present as a lactam, a ring-opened hydrolyzed lactam, or a combination thereof.

Certain substituted compounds Formula A and Formula I (and the subformula thereof) have one or more stereogenic centers. In certain embodiment thereof, such compounds may be enantiomers, and may have an enantiomeric excess of at least 55%. Within further embodiments thereof, such compounds have an enantiomeric excess of at feast 60%, 70%, 80%, 85%, 90%, 95%, 98%, or 99%. Certain compounds having one or more stereogenic centers have a enantiomeric excess of at least 99%.

Certain compounds of Formula A and Formula I (and the subformulae thereof) have two or more stereogenic centers. In certain embodiments thereof, such compounds have a diastereomeric excess of at least 55%. In other embodiments thereof such compounds have a diastereomeric excess of 60%, 70%, 80%, 85%, 90%, 95%, or 98%. Certain compounds having two or more stereogenic centers have a diastereomeric excess of at least 99%.

For detection purposes, compounds provided herein may be isotopically-labeled or radiolabeled. Accordingly, compounds recited in Formula I (or any other formula specifically recited herein) may have one or more atoms replaced by an atom of the same element having an atomic mass or mass number different from the atomic mass or mass number usually found in nature. Examples of isotopes that can be present in compounds provided herein include isotopes of hydrogen, carbon, nitrogen, oxygen, phosphorous, fluorine and chlorine, such as ²H, ³H, ¹¹C, ¹³C, ¹⁴C, ¹⁵N, ¹⁸O, ¹⁷O, ³¹P, ³²P, ³⁵S, ¹⁸F and ³⁶Cl. In addition, substitution with heavy isotopes such as deuterium (i.e., 2H) can afford certain therapeutic advantages resulting from greater metabolic stability, for example increased in vivo half-life or reduced dosage requirements and, hence, may be preferred in some circumstances.

Therapeutic Methods of Use

Compounds of the present invention, such as compounds of Formula I and I-A are useful as pharmaceuticals for the treatment of mammals, including humans, particularly for the treatment of mammals having a viral or retroviral infection such as an immunodeficiency disorder and/or are HIV positive, particularly a human who is HIV positive or is suffering from or susceptible to AIDS. Compounds of the invention may combat retroviral infections by inhibiting or arresting retroviral replication pathways.

Thus, the invention provides a method for the treatment of retroviral infections, particularly for the treatment of AIDS, in mammals including humans. The method comprising administration of an effective amount of one or more compounds of the invention in a pharmaceutically useful form, once or several times a day or other appropriate schedule, orally, rectally, parenterally particularly intravenously), topically, etc.

For such treatment, the compounds of the invention are administered in effective amounts and in appropriate dosage form ultimately at the discretion of the medical or veterinary practitioner. For example, as known to those skilled in the art, the amount of compounds of the invention required to be pharmaceutically effective will vary with a number of factors such as the mammal's weight, age and general health, the efficacy of the particular compound and formulation, route of administration, nature and extent of the condition being treated, and the effect desired. The total daily dose may be given as a single dose, multiple doses, or intravenously for a selected period. Efficacy and suitable dosage of a particular compound can be determined by known methods which follow. More particularly, for treatment of a tumor in a mammal such as a human, particularly when using more potent compounds of the invention, a suitable effective dose of the N-aryl retinamide will be in the range of 0.1 to 100 milligrams per kilogram body weight of recipient per day, preferably in the range of 1 to 10 milligrams per kilogram body weight of recipient per day. The desired dose is suitably administered once daily, or as several sub-doses, e.g. 2 to 4 sub-doses administered at appropriate intervals through the day, or other appropriate schedule. Such sub-doses may be administered as unit dosage forms, e.g., containing from 0.2 to 200 milligrams of compound(s) of the invention per unit dosage, preferably from 2 to 20 milligrams per unit dosage.

The therapeutic compound(s) may be administered alone, or as part of a pharmaceutical composition, comprising at least one N-aryl retinamide compound suitable for use in the methods of the invention together with one or more acceptable carriers thereof and optionally other therapeutic ingredients, e.g., other antiviral, antiretroviral AIDS agents or part of a cocktail of therapeutic agents. The carrier(s) must be “acceptable” in the sense of being compatible with the other ingredients of the formulation and not deleterious to the recipient thereof.

The compositions include those suitable for oral, rectal, nasal, topical (including buccal and sublingual), vaginal or parenteral (including subcutaneous, intramuscular, intravenous and intradermal) administration. The formulations may conveniently be presented in unit dosage form, e.g., tablets and sustained release capsules, and in liposomes, and may be prepared by any methods well known in the art of pharmacy.

Such methods include the step of bringing into association the to be administered ingredients with the carrier which constitutes one or more accessory ingredients. In general, the compositions are prepared by uniformly and intimately bringing into association the active ingredients with liquid carriers, liposomes or finely divided solid carriers or both, and then if necessary shaping the product.

The present invention further provides methods for treating patients suffering from a viral or retroviral infection, particularly retroviral infection such as retroviral infections capable of causing autoimmune diseases. Preferred methods of treatment of the invention are for treating HIV positive patients or patients suffering from or susceptible to AIDS. As used herein, the term “treatment” encompasses both disease-modifying treatment and symptomatic treatment, either of which may be prophylactic (i.e., before the onset of symptoms, in order to prevent, delay or reduce the severity of symptoms) or therapeutic (i.e., after the onset of symptoms, in order to reduce the severity and/or duration of symptoms).

Pharmaceutical Preparations

The present invention also provides pharmaceutical compositions comprising one or more N-aryl retinamide compounds according to Formula I, together with at least one physiologically acceptable carrier or excipient. Pharmaceutical compositions may comprise, for example, one or more of water, buffers (e.g., neutral buffered saline or phosphate buffered saline), ethanol, mineral oil, vegetable oil, dimethylsulfoxide, carbohydrates (e.g., glucose, mannose, sucrose or dextrans), mannitol, proteins, adjuvants, polypeptides or amino acids such as glycine, antioxidants, chelating agents such as EDTA or glutathione and/or preservatives. As noted above, other active ingredients may (but need not) be included in the pharmaceutical compositions provided herein.

A carrier is a substance that may be associated with an active compound prior to administration to a patient, often for the purpose of controlling stability or bioavailability of the compound. Carriers for use within such formulations are generally biocompatible, and may also be biodegradable. Carriers include, for example, monovalent or multivalent molecules such as serum albumin (e.g., human or bovine), egg albumin, peptides, polylysine and polysaccharides such as aminodextran and polyamidoamines. Carriers also include solid support materials such as beads and microparticles comprising, for example, polylactate polyglycolate, poly(lactide-co-glycolide), polyacrylate, latex, starch, cellulose or dextran. A carrier may bear the compounds in a variety of ways, including covalent bonding (either directly or via a linker group), noncovalent interaction or admixture.

Pharmaceutical compositions may be formulated for any appropriate manner of administration, including, for example, topical, oral, nasal, rectal or parenteral administration. In certain embodiments, compositions in a form suitable for oral use are preferred. Such forms include, for example, pills, tablets, troches, lozenges, aqueous or oily suspensions, dispersible powders or granules, emulsion, hard or soft capsules, or syrups or elixirs. Within yet other embodiments, compositions provided herein may be formulated as a lyophilizate. The term parenteral as used herein includes subcutaneous, intradermal, intravascular (e.g., intravenous), intramuscular, spinal, intracranial, intrathecal and intraperitoneal injection, as well as any similar injection or infusion technique.

Compositions intended for oral use may be prepared according to any method known to the art for the manufacture of pharmaceutical compositions and may contain one or more agents sweetening agents, flavoring agents, coloring agent, and preserving agents in order to provide appealing and palatable preparations. Tablets contain the active ingredient in admixture with physiologically acceptable excipients that are suitable for the manufacture of tablets. Such excipients include, for example, inert diluents (e.g., calcium carbonate, sodium carbonate, lactose, calcium phosphate or sodium phosphate), granulating and disintegrating agents (e.g., corn starch or alginic acid), binding agents (e.g., starch, gelatin or acacia) and lubricating agents (e.g., magnesium stearate, stearic acid or talc). The tablets may be uncoated or they may be coated by known techniques to delay disintegration and absorption in the gastrointestinal tract and thereby provide a sustained action over a longer period. For example, a time delay material such as glyceryl monosterate or glyceryl distearate may be employed.

Formulations for oral use may also be presented as hard gelatin capsules wherein the active ingredient is mixed with an inert solid diluent (e.g., calcium carbonate, calcium phosphate or kaolin), or as soft gelatin capsules wherein the active ingredient is mixed with water or an oil medium (e.g., peanut oil, liquid paraffin or olive oil).

Aqueous suspensions contain the active material(s) in admixture with excipients suitable for the manufacture of aqueous suspensions. Such excipients include suspending agents (e.g., sodium carboxymethylcellulose, methylcellulose, hydropropylmethylcellulose, sodium alginate, polyvinylpyrrolidone, gum tragacanth and gum acacia); and dispersing or wetting agents (e.g., naturally-occurring phosphatides such as lecithin, condensation products of an alkylene oxide with fatty acids such as polyoxyethylene stearate, condensation products of ethylene oxide with long chain aliphatic alcohols such as heptadecaethyleneoxycetanol, condensation products of ethylene oxide with partial esters derived from fatty acids and a hexitol such as polyoxyethylene sorbitol monooleate, or condensation products of ethylene oxide with partial esters derived from fatty acids and hexitol anhydrides such as polyethylene sorbitan monooleate). Aqueous suspensions may also comprise one or more preservatives, for example ethyl or n-propyl p-hydroxybenzoate, one or more coloring agents, one or more flavoring agents, and one or more sweetening agents, such as sucrose or saccharin. Syrups and elixirs may be formulated with sweetening agents, such as glycerol, propylene glycol, sorbitol, or sucrose. Such formulations may also comprise one or more demulcents, preservatives, flavoring agents, and/or coloring agents.

Oily suspensions may be formulated by suspending the active ingredients in a vegetable oil (e.g., arachis oil, olive oil, sesame oil, or coconut oil) or in a mineral oil such as liquid paraffin. The oily suspensions may contain a thickening agent such as beeswax, hard paraffin, or cetyl alcohol. Sweetening agents, such as those set forth above, and/or flavoring agents may be added to provide palatable oral preparations. Such suspensions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

Pharmaceutical compositions may also be in the form of oil-in-water emulsions. The oily phase may be a vegetable oil (e.g., olive oil or arachis oil), a mineral oil (e.g., liquid paraffin), or a mixture thereof. Suitable emulsifying agents include naturally-occurring gums (e.g., gum acacia or gum tragacanth), naturally-occurring phosphatides (e.g. soy-bean, lecithin, and esters or partial esters derived from fatty acids and hexitol), anhydrides (e.g., sorbitan monoleate), and condensation products of partial esters derived from fatty acids and hexitol with ethylene oxide (e.g., polyoxyethylene sorbitan monoleate). An emulsion may also comprise one or more sweetening and/or flavoring agents.

The pharmaceutical composition may be prepared as a sterile injectible aqueous or oleaginous suspension in which the modulator, depending on the vehicle and concentration used, is either suspended or dissolved in the vehicle. Such a composition may be formulated according to the known art using suitable dispersing, wetting agents and/or suspending agents such as those mentioned above. Among the acceptable vehicles and solvents that may be employed are water, 1,3-butanediol, Ringer's solution and isotonic sodium chloride solution. In addition, sterile, fixed oils may be employed as a solvent or suspending medium. For this purpose any bland fixed oil may be employed, including synthetic mono- or diglycerides. In addition, fatty acids such as oleic acid may be used in the preparation of injectible compositions, and adjuvants such as local anesthetics, preservatives and/or buffering agents can be dissolved in the vehicle.

Pharmaceutical compositions may be formulated as sustained release formulations (i.e., a formulation such as a capsule that effects a slow release of modulator following administration). Such formulations may generally be prepared using well known technology and administered by, for example, oral, rectal, or subcutaneous implantation, or by implantation at the desired target site. Carriers for use within such formulations are biocompatible, and may also be biodegradable; preferably the formulation provides a relatively constant level of modulator release. The amount of a N-aryl retinamide compound according to Formula I contained within a sustained release formulation depends upon, for example, the site of implantation, the rate and expected duration of release and the nature of the viral infection to be treated or prevented.

N-aryl retinamide compounds provided herein are generally administered in an amount that achieves a concentration in a body fluid (e.g., blood, plasma, serum, CSF, synovial fluid, lymph, cellular interstitial fluid, tears or urine) that is sufficient to detectably inhibit the formation of (glyco)sphingolipid domains on the surface of target cell membranes and thereby prevent or inhibit viral infection. A dose is considered to be effective if it results in a discernible patient benefit as described herein. Preferred systemic doses range from about 0.1 mg to about 140 mg per kilogram of body weight per day (about 0.5 mg to about 7 g per patient per day), with oral doses generally being about 5-20 fold higher than intravenous doses. The amount of active ingredient that may be combined with the carrier materials to produce a single dosage form will vary depending upon the host treated and the particular mode of administration. Dosage unit forms will generally contain between from about 1 mg to about 500 mg of an active ingredient.

Pharmaceutical compositions may be packaged for treating conditions responsive to viral infections, retroviral infections, HIV positive patients or patients suffering from or susceptible to AIDS. Packaged pharmaceutical compositions may include a container holding a effective amount of at least one one N-aryl retinamide compound as described herein and instructions (e.g., labeling) indicating that the contained composition is to be used for treating a viral infection responsive to one N-aryl retinamide compound administration in the patient

All documents mentioned herein are incorporated herein by reference.

### EXAMPLES

### Materials and Methods

Reagents and Cells:

The TMZ cell line was obtained through the AIDS Research and Reference Reagent Program, Division of AIDS, National Institute of Allergy and infectious Diseases, National Institutes of Health. This indicator cell line is a HeLa cell line derivative that expresses high levels of CD4 and CCR5 along with endogenously expressed CXCR4. TMZ cells contain HIV LTR-driven β-galactosidase and luciferase reporter cassettes that are activated by HIV tat expression. TMZ cells were routinely subcultured every 3 to 4 days by trypsinization and were maintained in DMEM supplemented with 10% fetal bovine serum and 1× penicillin-streptomycin (complete media). The infectious titer of all virus stocks was determined on TMZ cells by direct counting of blue foci. HeLa cells expressing different levels of CD4 and coreceptor were gifts from David Kabat (Oregon Health Sciences University). HeLa cells were grown in Dulbecco modified Eagle medium plus 10% fetal bovine serum (FBS). HIV-1 envelope proteins were transiently expressed on the surface of HeLa cells with the recombinant vaccinia virus constructs vPE16 (IIIB, CXCR4 utilizing and (Ba-L, CCR5 utilizing) as described.

Shingomyelinase derived from Bacillus cereus, etoposide, all trans retinoic acid, and daunorubicin were obtained from Sigma. HPR was purchased from Biomol research labs.

Infectivity Assay:

TMZ cells (2×10⁴ per well) were added to 96-well microtiter plate wells (Falcon, Lincon Park, N.J.) in 100 μl of complete media and allowed to adhere 15-18 hours at 37° C. An equivalent amount of each virus stock (MOI of 0.01) was added to the cell monolayers in the presence of 40 μg/ml DEAE-dextran in DMEM in a final volume of 100 μL. Viral infection was allowed to proceed for 2 hr at 37° C. following which, 100 μl of complete DMEM media was added. Luciferase activity was measured after 15-18 hours at 37° C. with 5% CO₂ in a humidified incubator using a Promega (Madison, Wis.) luciferase assay system kit. Briefly, the supernatants were removed and the cells were lysed with Steady Glo luciferase assay system. The light intensity of each well was measured on a Reporter luminometer. Mock-infected cells were used to determine background luminescence. All infectivity assays were performed in duplicate.

Activation of the β-galactosidase gene was detected by fixing the cells in 0.25% glutaraldehyde-0.8% formaldehyde in PBS for 5 min at room temperature, washed three times in PBS and subsequently stained with a solution containing 400 μg of X-gal (5-bromo-4-chloro-3-indolyl-B-D-galactopyranoside) per ml, 4 mM MgCl₂, 4 mM potassium ferrocyamide, and 4 mM potassium ferricyanide in PBS overnight at 37° C. The staining solution was then removed and the cells were overlaid in PBS to allow for microscopic analysis.

Cell-Cell Fusion Assay:

Target TMZ cells were plated at 0.75×10⁴ cells per well in a 96 well plate and treated with a 4-BPR compound for 48 hours with 5% CO₂ in a humidified incubator. HeLa cells expressing HUV-1 gp160 (HXB2) in addition to gag, tat, rev and nef were then cocultured with target cells containing the tat inducible luciferase reporter cassette. Envelope and target cells were cocultivated at 37° C. for 7 hours. Luciferase activity was then measured using a Promega (Madison, Wis.) luciferase assay system kit. Briefly, the supernatants were removed and the cells were lysed with Steady Glo luciferase assay system. The light intensity of each well was measured on a Reporter luminometer. Mock-infected cells were used to determine background luminescence. All infectivity assays were performed in duplicate.

Flow Cytometry:

TMZ cells, harvested with trypsin-EDTA in PBS, were centrifuged at 450×g and resuspended at 10⁶ cells/ml in PBS-5% FBS-5% normal mouse serum. After incubation for 15 min at room temperature, the cells were washed twice in PBS-0.1% bovine serum albumin and resuspended in 100 μl of PBS-5% FBS-5% normal mouse serum. 20 μl of Phycoerythrin (PE)-conjugated mouse immunoglobulin G (IgG) anti-CD4, PE-conjugated mouse IgG anti-CXCR4, or PE-conjugated mouse IgG anti-CCR5, from Becton Dickenson (San Jose, Calif.) was then added to each sample. Cells were incubated at 4° C. for 1 hour and washed twice in PBS-0.1% BSA. Samples were fixed in PBS-1% paraformaldehyde and resuspended in 1 ml of PBS to be read by a FACScalibur instrument (Becton Dickinson, San Jose, Calif.) at 10,000 events/sample with respect to unlabeled cells.

Sphingomyelinase Treatment:

In order to induce cell surface ceramide, cells were incubated with purified sphingomyelinase at 50 mU/ml for 10 min at 37° C.

Exogenous ceramide addition:

100 μM C16 ceramide and C24 ceramide were sonicated for 5 min in 95% ethyl alcohol. 1 μl of each solution was dissolved in DMEM yielding a final concentration of 1 μM lipid. Cells were then treated for 10 min with this lipid solution. Following removal of the lipid, virus was added in 40 μg/1 ml DEAE-dextran/DMEM and the viral infectivity assay was carried out as previously described.

Treatment of Cell Cultures with [³H]Sphingosine:

5×10⁵ cells were seeded on 10 cm tissue culture plates. After 12 hours the cells were incubated with 1 μCi [³H] sphingosine (specific activity 20 Ci/mmol) (American Radiological Chemicals) in medium containing a 4-HEPR compound at final concentrations of 0, 2, 5 and 10 μM for up to 48 hours. Similar protocol was followed for ceramide estimation upon treatment with drugs such as PPMP, Daunorubicin and etoposide. For sphingomyeleinase treatment cells labeled with [³H]sphingosine were incubated with 50 mili units of Bacillus cereus Sphingomyeleinase (Sigma) in 5 ml PBS containing Ca⁺⁺ and Mg⁺⁺ and incubated for 10 minutes at 37° C. followed by washing with PBS containing Ca⁺⁺ and Mg⁺⁺.

Lipid Extraction and Ceramide Quantitation:

After 48 hours cells harvested with cell dissociation buffer containing EDTA in phosphate-buffered saline (PBS) from Invitrogen, were pelleted at 450×g for 5 min. Lipids were then extracted according to Bligh and Dyer. Briefly, the cell pellet (10⁶ cells) was suspended in 0.5 ml of H₂O, which was added to 2 ml of CH₃OH:CHCl₃ (2:1, vol/vol). After vortexing, 0.5 ml CHCl₃ and 0.5 ml H₂O were added, the suspension was vortexed and centrifuged at 100×g for 5 min to separate the two phases. The extract in the lower phase was then removed for storage, and the CHCl₃-H₂O step was repeated twice with the aqueous phase. Extracted lipid phase were pooled, dried under N₂, resuspended in CH₃OH:CHCl₃ (2:1, vol/vol). A small amount of the extract from each treatment was used for normalization. After normalization, the lipid extracts labeled with [³H]sphingosine were run on TLC. The TLC was developed in solvent system comprising CHCl₃:CH₃OH:Water (65:25:5, vol/vol/vol). The TLC plates were then sprayed lightly with En³hance (Perkin Elner), and radioactive lipids were visualized by autoradiography after 48 h at −80° C. The radioactive spot migrating with the ceramide standard was scraped from the plate and quantified by liquid scintillation spectrometry.

### Example 1

### Pharmacological Stimulation of Ceramide Synthesis Inhibits HIV-1 Infection

The effects on overexpression of ceramide, a highly hydrophobic lipid with membrane restructuring properties, were evaluated on HIV infectivity. Initially, a variety of pharmacological agents that have been shown to increase de novo ceramide synthesis were selected and the effect of these agents on HIV-1 infection were investigated. A viral-cell fusion system that utilizes an indicator cell line that has been engineered to express CD4 and CCR5 was employed. As CXCR4 is endogenously expressed on this cell line it is susceptible to infection by diverse HIV isolates. Following viral fusion the LTR driven reported gene products luciferase and β-galactosidase are expressed allowing for quantitative measurement of viral infectivity as soon as 16 h post infection. Such an assay system allows for the determination of viral-cell fusion inhibition as well as inhibition of early HIV lifecycle events. Four agents that target ceramide biosynthesis, a 4-HPR compound (fenretinide, N-(4-hydroxyphenyl)retinamide), all trans retinoic acid, etoposide and daunorubicin, were employed.

FIG. 1 illustrates the pathways involved in ceramide metabolism and agents that target specific enzymes involved in this process. a 4-HPR compound, a synthetic derivative of retinoic acid, specifically increased the generation of de novo ceramide through the activation of two key enymes in the ceramide synthesis pathway, serine palmitoyltransferase and ceramide synthase. Etoposide is an activator of serine palmitoyltransferase, while daunorubicin activates ceramide synthase. Retinoic Acid has also been reported to upregulate ceramide levels due to activation of serine palmitoyltransferase (J. Biol. Chem., Vol. 275, Issue 39, 30344-30354, Sep. 29, 2000).

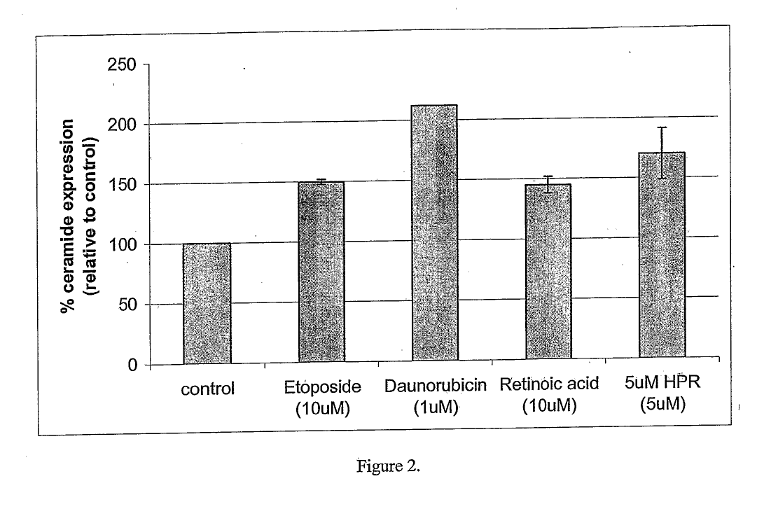

Treatment of cells with all four agents up-regulates ceramide as seen in FIG. 2. Ceramide levels were determined following incorporation of [³H] sphingosine for 48 hours in the presence of the appropriate agent. Analysis of the potential apoptotic effects of these agents on the cells by annexin 5 staining, revealed that at the concentrations employed in these assays such an effect is not observed.

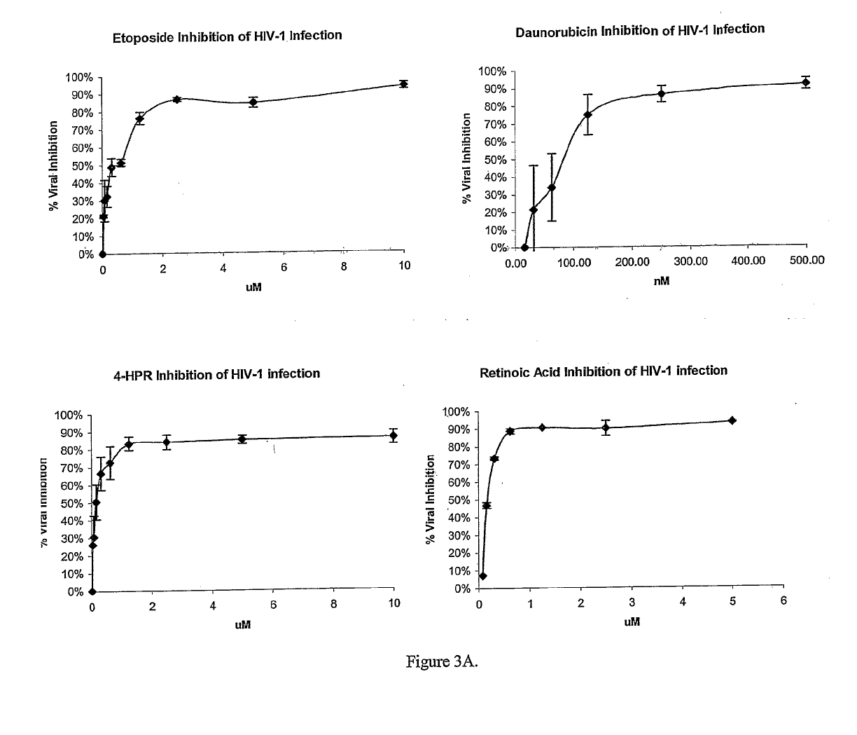

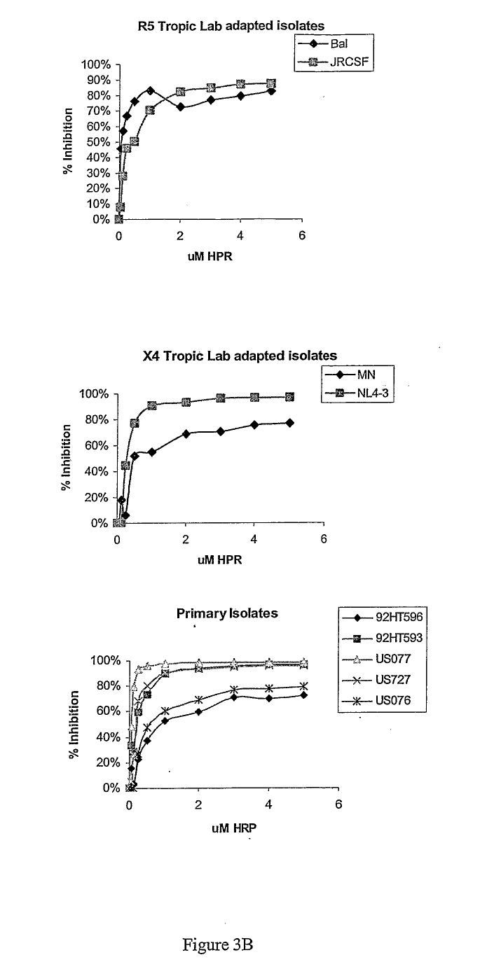

Treatment of target cells with these agents at the indicated concentrations for 48 hours resulted in a significant dose dependent inhibition of MN, an X4 tropic HIV-1 isolate (FIG. 3a). a 4-HPR compound, which targets both serine palmitoyltranferase and ceramide synthase, was selected to probe the breadth of HIV-1 isolates that are inhibited upon upregulation of ceramide levels. Following treatment of target cells with a 4-HPR compound a dose-dependent inhibition of X4 (NL4-3, MN), R5 (Bal, JRCSF) and primary isolates was observed (FIG. 3b). In contrast to the potent inhibitory effect of a 4-HPR compound on HIV-1, infectivity of VSV pseudotyped HIV-1 was not impaired under a 4-HPR compound concentrations less than 5 μM.

As inhibition of HIV-1 is observed at low μl concentrations for a 4-HPR compound, with minimal toxicity these results have important consequences for HIV therapy. Indeed, the IC₅₀ for most isolates tested is less than 1 μM, concentrations that are attainable in vivo with a 4-HPR compound under therapeutic conditions.

### Example 2

### Enzymatic Generation of Ceramide at the Plasma Membrane Inhibits Viral Infection

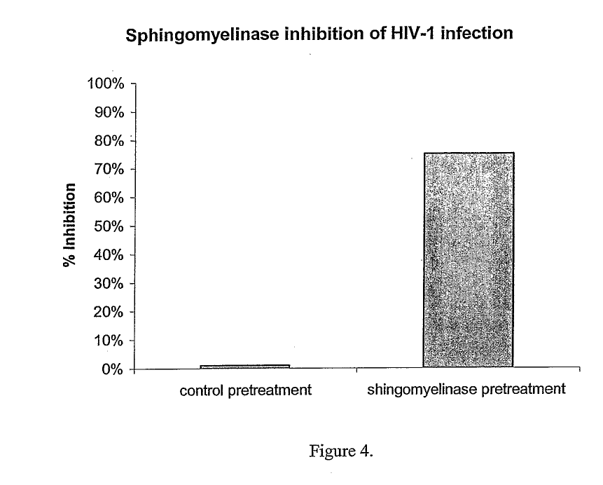

To determine if increasing ceramide directly at the plasma membrane could inhibit HIV infection, cells were pretreated for 10 minutes at 37° C. with sphingomyelinase. This enzyme cleaves sphingomyelin, a phospholipid mainly located on the outer leaflet of the plasma membrane, into ceramide. Evidence indicates that sphingomyelin is localized in preformed triton insoluble microdomains termed “rafts” in the plasma membrane, which would then be the site where ceramide is generated. Prior analysis in liposomes and lipid monolayers has indicated that such formation of ceramide possibly drive structural reorganization of membrane lipids. When sphingomyelinase was employed to pretreat target cells, 60-70% inhibition of HIV-1 infection was observed (FIG. 4). Analysis of ceramide expression in these cells confirmed that ceramide levels were increased following enzymatic activity.

### Example 3

### Exogenous Ceramide Inhibits HIV-1 Infection

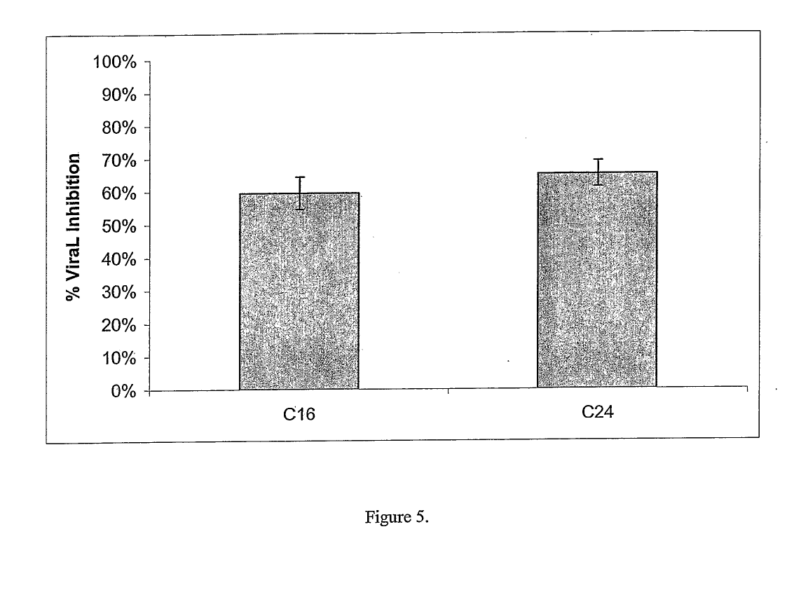

To further confirm that ceramide accumulation at the plasma membrane inhibits HIV-1 infection exogenous ceramide was added directly to target cells prior to infection. Two long chain ceramides were selected, C16 and C24, which most likely mimic the hydrophobic and partition properties of endogenous ceramide. Pretreatment of target cells with 1 μM C16 or a mixture of C16 and C24 for 10 minutes at 37° C. resulted in sufficient ceramide accumulation to inhibit HIV infection approximately 60% (FIG. 5). These results confirm that increasing ceramide concentrations result in an inhibition to HIV-1 infection.

### Example 4

### Pharmacological Modulation of Ceramide Levels Inhibits HIV-1 Infection in Primary Monocytes Derived Macrophages

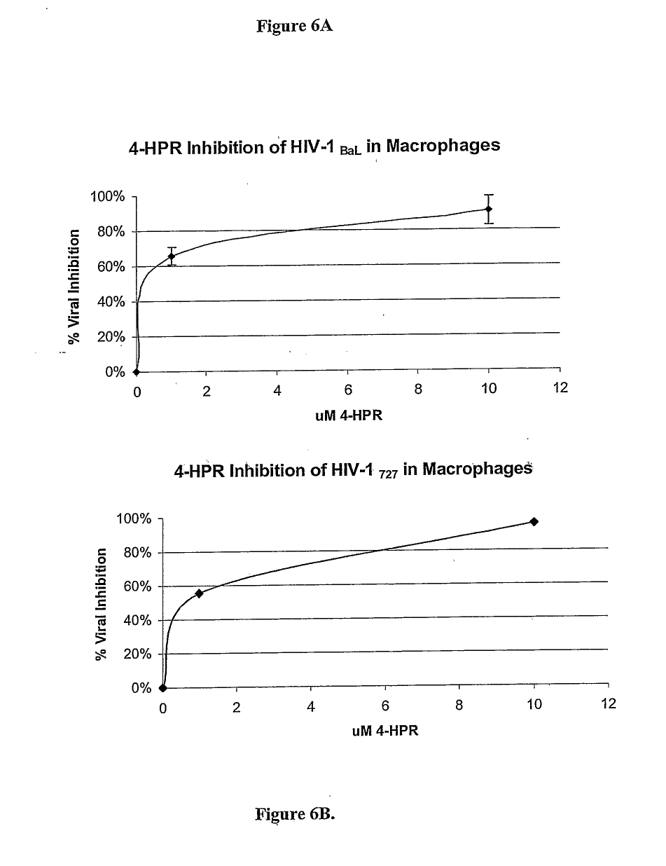

Having established that increasing ceramide levels has inhibitory consequences for viral entry in an epithelial carcinoma cell line (HeLa), we next determined the effects of such perturbation on HIV-1 infection of primary cells. Again, a 4-HPR compound was employed to increase ceramide levelsand differentiated macrophages were pretreated with a 4-HPR compound for 2 days. Following treatment with 5 uM of a 4-HPR compound monocyte derived macrophages show a 1.12-fold increase in ceramide, which is substanitially less than what is observed in HeLa cells following such treatment (1.7-fold). However, when a 4-HPR compound treated macrophages were subjected to HIV-1 infection they proved to be resistant to HIV-1 infection at low concentrations of a 4-HPR compound (FIGS. 6A and 6B). Infection inhibition was observed at low uM concentrations for both a CCR5 tropic isolate (BaL) (FIG. 6A) and for a primary isolate (FIG. 6B).

### Example 5

### Activation of Ceramide Synthesis Downmodulates HIV-1 Receptors

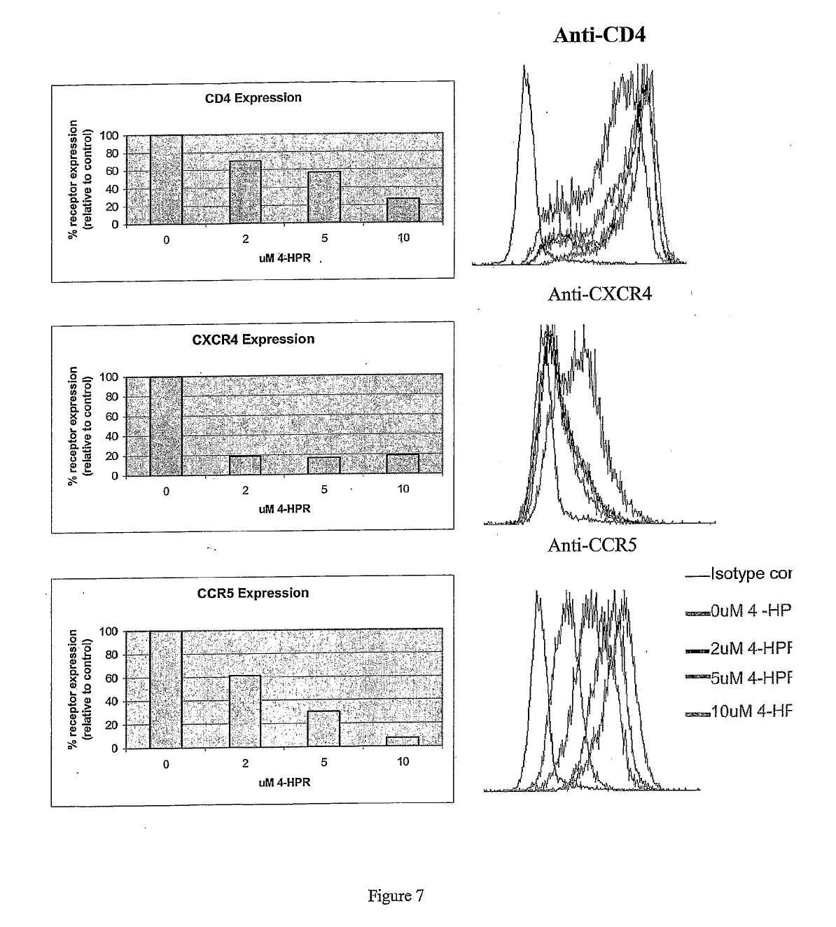

Increasing ceramide concentrations is thought to perturb membrane properties due to the hydrophobic nature of this lipid and the tendency to self aggregate. The possible consequences of membrane altering properties on the cell surface expression of the main HIV receptors, CD4, X4 and R5, was investigated. As seen in FIG. 7, treatment with a 4-HPR compound results in downregulation of all three receptors. CD4 expression decreases in a dose dependent manner from a control level of 100% to levels of 70%, 57% and 27% following treatment with 2, 5 and 10 μM of a 4-HPR compound respectively. Likewise CCR5 expression is similarly decreased to levels of 60%, 30% and 7% following treatment with 2, 5 and 10 μM of a 4-HPR compound respectively. CXCR4 shows a more dramatic decrease in expression levels, decreasing to 18% following treatment with 2 μM of a 4-HPR compound. Thus, a 4-HPR compound, and other pharmacological agents that target ceramide synthesis, may potentially inhibit HIV-1 infection at the level of membrane fusion by decreasing the availability of the primary receptor CD4 and/or the necessary coreceptors X4 and R5.

### Example 6

### Activation of Ceramide Synthesis Inhibits Cell-Cell Fusion

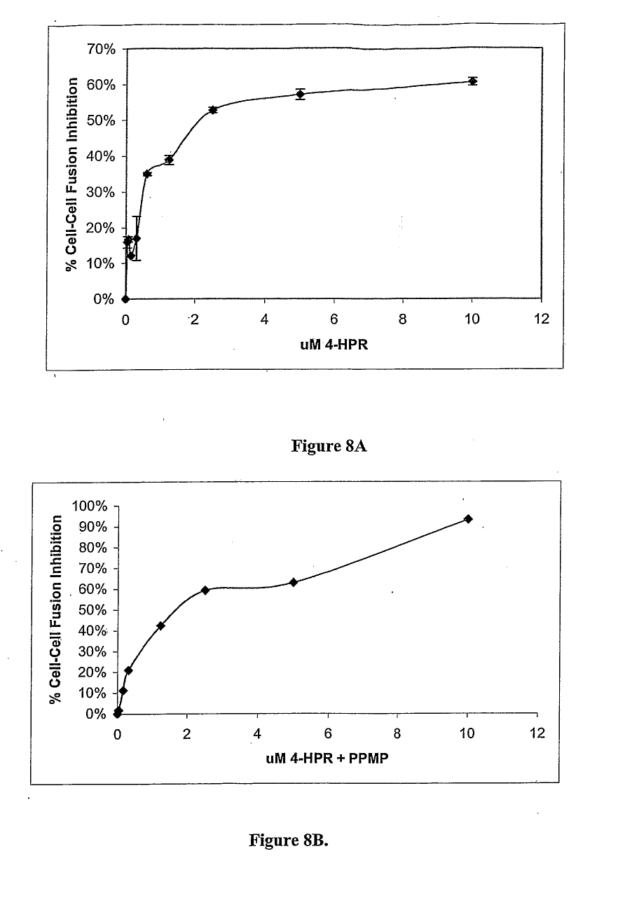

To investigate the possibility that ceramide upregulation inhibits viral infection at the level of membrane fusion we employed a well characterized reporter fusion assay. Briefly, HeLa cells expressing HIV-1 gp160 (HXB2) in addition to gag, tat, rev and nef were cocultured with CD4 positive target cells containing the tat inducible luciferase reporter cassette. Target cells were pretreated with a 4-HPR compound for 2 days with subsequent cocultivation with envelope expressing cells. Envelope and target cells were cocultivated at 37° C. for 7 hours following which the cells were lysed and luciferase activity quantitated. 50% inhibition of cell-cell fusion was observed with target cells treated with 2.5 μM of a 4-HPR compound, which increased to 60% inhibition upon treatment with 10 μM of a 4-HPR compound (FIG. 8A).

### Example 7

### Inhibition of Glucosylceramide Synthase

Although cell-cell fusion systems have been widely used as a model for viral-cell fusion mechanisms, the merging of two cell membranes may be a more robust process than the merging of viral and cell membranes, especially in systems where envelope and receptor proteins are overexpressed. To further increase ceramide concentrations an inhibitor of glucosylceramide synthase was used, which prevents the shunting of ceramide through the glycolipid synthesis pathway. It was reasoned that inhibiting GlcCer synthase should result in an augmentation of ceramide concentrations in the cell possibly revealing a block to membrane fusion. Analysis of ceramide concentrations through incorporation of [3H] sphingosine revealed that combining an activator of ceramide synthesis (a 4-HPR compound) with an inhibitor of GicCer synthase (PPMP) substantially augments cellular ceramide levels. When both agents were combined to pretreat target cells a substantial (75%) inhibition of fusion was observed (FIG. 8B). A 4-HPR compound alone demonstrated a slight inhibition of fusion (20%) while PPMP was ineffective. These results implicate ceramide accumulation in inhibiting membrane fusion possibly highlighting the mechanism of action of HIV-1 inhibition in our viral infectivity assay.

### Example 8

### Inhibition of Viral Fusion

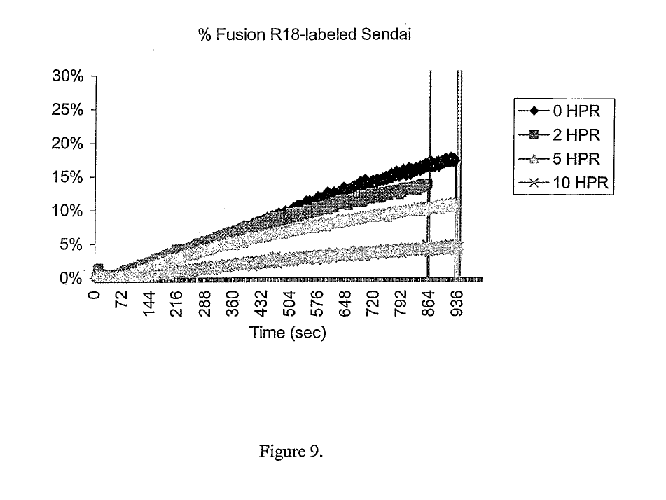

To further probe how ceramide accumulation might inhibit viral fusion, viral fusion was monitored using a well-established octadecyl rhodamine (R18) fluorescence dequenching (R18-fdq) assay. In this assay system R18-labeled Sendai virus was incubated with control or a 4-HPR compound treated cells for 30 min at 4° C. to allow for virus binding. Small aliquots of virus-cell complexes were added to a stirred cuvette at 37° C. containing 2 ml buffer at pH7. Fluorescence increases were recorded with a 1 sec time resolution in a SLM8000 spectrofluorimeter (SLMInstruments, Urbana, Ill.). As seen in FIG. 9, a slow increase in fluorescence was observed for untreated control cells following a brief lag period as a result of viral fusion. This increase in fluorescence reflects dequenching of R18 upon the merging of viral and cell membranes. For sendai virus there is not a concerted trigger for fusion as is observed for other viruses such as influenza upon protonation of the envelope protein. Hence, fluorescence dequenching indicative of sendai virus fusion occurs over a long time frame. As seen in FIG. 9, treatment of target cells with increasing concentrations of a 4-HPR compound results in a dose dependent inhibition of viral fusion as reflected by diminished fluorescence dequenching.

The foregoing description is illustrative thereof, and it will be understood that variations and modifications can be effected without departing from the scope or spirit of the invention as set forth in the following claims.

1. A method of preventing or inhibiting a viral infection in a subject, the method comprising administering to said subject a pharmaceutical composition comprising a ceramide-generating retinoid or a pharmaceutically acceptable salt thereof.

2. A method of preventing or inhibiting a viral infection in a subject, the method comprising administering to said subject a pharmaceutical composition comprising a ceramide-degradation inhibitor or a pharmaceutically acceptable salt thereof.

3. A method of preventing or inhibiting a viral infection in a subject, the method comprising administering to said subject a pharmaceutical composition comprising:
(a) a ceramide-generating retinoid or a pharmaceutically acceptable salt thereof; and
(b) a ceramide-degradation inhibitor or a pharmaceutically acceptable salt thereof.

4. A method of claim 1 wherein the ceramide-generating retinoid is a retinoic acid derivative.

5. A method of claim 1 wherein the ceramide degradation inhibitor is selected from the group consisting of glucosyl ceramide synthase inhibitors, sphingosine-1-phosphate synthesis inhibitors, protein kinase C inhibitors, and the pharmaceutically acceptable salts thereof.

6. A method of preventing or inhibiting a viral infection, the method comprising administering a pharmaceutical composition comprising at least one N-(aryl)retinamide compounds to the subject suffering from or susceptible to a viral infection.

7. The method of claim 6, wherein the N-(aryl)retinamide modulates ceramide metabolism.

8. The method of claim 6, wherein the pharmaceutical composition comprises N-(4-hydroxyphenyl)retinamide or a derivative thereof.

9. The method of claim 6, wherein the pharmaceutical composition comprises at least one compound of the formula: 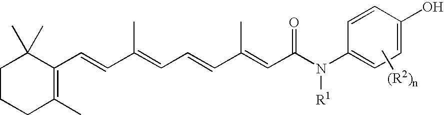
wherein:
R¹ is hydrogen, optionally substituted alkyl, optionally substituted alkenyl, optionally substituted alkynyl, or optionally substituted aralkyl;
R² is independently selected at each occurrence from the group consisting of hydrogen, halogen, hydroxy, optionally substituted alkoxy, optionally substituted alkyl, optionally substituted alkenyl, optionally substituted alkynyl, optionally substituted amino, and optionally substituted mono- and di-alkylamino; and
n is an integer of from 0 to about 4.

10. The method of claim 6, wherein the pharmaceutical composition comprises N-(4-hydroxyphenyl)retinamide.

11. The method of claim 9, wherein the pharmaceutical composition further comprises one or more therapeutic agents selected from 1-phenyl-2-hexadecanoylamino-3-morpholino-1-propanol, chemokine inhibitors, H IV fusion inhibitors, viral protease inhibitors, reverse transcriptase inhibitors, and entry inhibitors.

12. The method of claim 6, wherein the subject is a mammal.

13. The method of claim 6, wherein the subject is a primate.

14. The method of claim 6, wherein the subject is a human.

15. The method of claim 6, wherein the N-(aryl)retinamide compound inhibits HIV infectivity at a concentration of less than 10 μM.

16. The method of claim 6, wherein the N-(aryl)retinamide compound inhibits HIV infectivity at a concentration of less than 5 μM.

17. A method of inhibiting HIV infectivity in a subject, the method comprising administration of N-(4-hydroxyphenyl)retinamide or a derivative thereof sufficient to increase ceramide levels in a cellular membrane susceptible to HIV entry

18. The method of claim 17, wherein the N-(4-hydroxyphenyl)retinamide or a derivative thereof decreases the viral load in a subject by about 40%.

19. A method of inhibiting HIV infectivity in a subject, the method comprising administration of compound that stimulates the de novo synthesis of ceramide sufficient to increase ceramide levels in a cellular membrane susceptible to HIV entry.

20. The method of claim 19, wherein the compound that stimulates the generation of ceramide is sphingomyelinase.

21. The method of claim 19, wherein sphingomyelinase decreases viral load in a subject by about 40%, at least about 50%, 60%, 75%, 80%, 99.9%, up to about 100%.

22. The method of claim 21, wherein viral load is due to infection by HIV.

23. A method of inhibiting a viral attachment/entry or exit phase of a virus by administering a pharmaceutical composition to a cell susceptible to infection by a virus, wherein the pharmaceutical composition comprises an inhibitor of at least one enzyme essential to ceramide metabolism.

24. The method of claim 23, wherein the enzyme is essential to a glycosylation step of ceramide metabolism.

25. The method of claim 23, wherein the pharmaceutical composition comprises at least one compound of the formula: 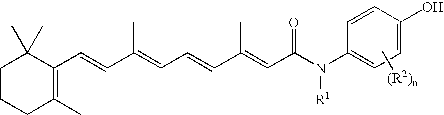
wherein:
R¹ is hydrogen, optionally substituted alkyl, optionally substituted alkenyl, optionally substituted alkynyl, or optionally substituted aralkyl;
R² is independently selected at each occurrence from the group consisting of hydrogen, halogen, hydroxy, optionally substituted alkoxy, optionally substituted alkyl, optionally substituted alkenyl, optionally substituted alkynyl, optionally substituted amino, and optionally substituted mono- and di-alkylamino; and
n is an integer of from 0 to about 4.

26. The method of claim 25, wherein the pharmaceutical composition comprises N-(4-hydroxyphenyl)retinamide.

27. The method of claim 26, wherein the pharmaceutical composition further comprises one or more therapeutic agents selected from 1-phenyl-2-hexadecanoylamino-3-morpholino-1-propanol, chemokine inhibitors, HIV fusion inhibitors, viral protease inhibitors, reverse transcriptase inhibitors, and entry inhibitors.

28. The method of claim 25, wherein the cell is a mammalian cell.

29. The method of claim 28, wherein the cell is an immune cell.

30. The method of claim 25, wherein the RNA virus is HIV.

31. The method of claim 25, wherein the N-(aryl)retinamide compound inhibits HIV infectivity at a concentration of less than 10 μM.

32. The method of claim 25, wherein the N-(aryl)retinamide compound inhibits HIV infectivity at a concentration of less than 5 μM.

33. The method of claim 25, wherein sphingomyelinase inhibits the viral attachment/entry phase of an RNA virus in a cell by about 40%, at least about 50%, 60%, 75%, 80%, 99.9%, up to about 100%.

34. A kit comprising:
a) one or more agents for increasing ceramide concentration of a cell,
b) means for detecting at least one of a) ceramide concentration of the cells, and 2) inhibition of viral infectivity of the cell; and
c) directions for using the kit.

35. The kit of claim 34, wherein the agents comprise a pharmaceutical composition of a N-aryl retinamide compound capable of activating ceramide biosynthesis in addition to a pharmaceutical composition that inhibits ceramide glycosolation and (glyco)sphingolipid formation.

36. The kit of claim 34, wherein the agents comprise any one or more of compositions as identified by Formula I and substituted groups thereof.
